SEQUENCE LISTING (39 sequences)

SEQ ID NO: 1 - protein, 121 aa, Artificial Sequence
  Synthetic polypeptide
QVTLKESGPGILQPSQTLSLTCSFSGFSLSTFGMGVGWIRQPSGKGLEWLAHIWWDDYKYYNPALKSRLTISKDTSKNQVFLKIANVDTADTATYYCARISYYGSSYLDYWGQGTTLTVSS

SEQ ID NO: 2 - protein, 113 aa, Artificial Sequence
  Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQTTHVPYTFGGGTKLEIKR

SEQ ID NO: 3 - protein, 60 aa, Artificial Sequence
  Synthetic polynucleotide
GGTTTGGCTGGCGGGCCCCTAGGAATGCTCTCCTCCCCGCCCCCGGCCCAAAGTGCCACG

SEQ ID NO: 4 - protein, 8 aa, Artificial Sequence
  Synthetic polypeptide
GFTFSNYW

SEQ ID NO: 5 - protein, 10 aa, Artificial Sequence
  Synthetic polypeptide
IRLRSDDYAT

SEQ ID NO: 6 - protein, 13 aa, Artificial Sequence
  Synthetic polypeptide
TGPANWGYWYFDV

SEQ ID NO: 7 - protein, 11 aa, Artificial Sequence
  Synthetic polypeptide
QSIVQTNGNTY

SEQ ID NO: 8 - protein, 3 aa, Artificial Sequence
  Synthetic polypeptide
KVS

SEQ ID NO: 9 - protein, 9 aa, Artificial Sequence
  Synthetic polypeptide
FQGSHVPLT

SEQ ID NO: 10 - protein, 8 aa, Artificial Sequence
  Synthetic polypeptide
GYTFTSYW

SEQ ID NO: 11 - protein, 8 aa, Artificial Sequence
  Synthetic polypeptide
IYPGNSDT

SEQ ID NO: 12 - protein, 15 aa, Artificial Sequence
  Synthetic polypeptide
TRRGYYYGSSSHFDY

SEQ ID NO: 13 - protein, 11 aa, Artificial Sequence
  Synthetic polypeptide
QSLVHSNGNTY

SEQ ID NO: 14 - protein, 3 aa, Artificial Sequence
  Synthetic polypeptide
KVS

SEQ ID NO: 15 - protein, 9 aa, Artificial Sequence
  Synthetic polypeptide
SQSTHVPPT

SEQ ID NO: 16 - protein, 122 aa, Artificial Sequence
  Synthetic polypeptide
EVKLEESGGGLVQPGGSMKLSCVASGFTFSNYWMNWVRQSPEKGLEWVAQIRLRSDDYATHYAESVKGRFTISRDDSQSSVYLQMNNLRAEDTGIYYCTGPANWGYWYFDVWGTGTTVTVSS

SEQ ID NO: 17 - protein, 112 aa, Artificial Sequence
  Synthetic polypeptide
DVLMTQTPLSLPVSLGDQASISCRSSQSIVQTNGNTYLEWYLQKPGQSPKLLIYKVSNRFSGVPDRFSGSGSGTDFTLKISRVEAEDLGVYYCFQGSHVPLTFGAGTKLELK

SEQ ID NO: 18 - protein, 122 aa, Artificial Sequence
  Synthetic polypeptide
EVQLQQSGTVLARPGASVKMSCKTSGYTFTSYWMHWVKQRPGQGLEWIGAIYPGNSDTSYNQKFKGKAKLTAVTSASTAYMELSSLTHEDSAVYYCTRRGYYYGSSSHFDYWGQGTTLTVSS

SEQ ID NO: 19 - protein, 112 aa, Artificial Sequence
  Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPTFGGGTKLEIK

SEQ ID NO: 20 - protein, 14 aa, Artificial Sequence
  Synthetic polypeptide
GGGGSGGGGSGGGG

SEQ ID NO: 21 - protein, 3 aa, Artificial Sequence
  Synthetic polypeptide
GGG

SEQ ID NO: 22 - protein, 227 aa, Homo Sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 731 aa, Artificial Sequence
  Synthetic polypeptide
EVKLEESGGGLVQPGGSMKLSCVASGFTFSNYWMNWVRQSPEKGLEWVAQIRLRSDDYATHYAESVKGRFTISRDDSQSSVYLQMNNLRAEDTGIYYCTGPANWGYWYFDVWGTGTTVTVSSGGGGSGGGGSGGGGSDVLMTQTPLSLPVSLGDQASISCRSSQSIVQTNGNTYLEWYLQKPGQSPKLLIYKVSNRFSGVPDRFSGSGSGTDFTLKISRVEAEDLGVYYCFQGSHVPLTFGAGTKLELKGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGEVQLQQSGTVLARPGASVKMSCKTSGYTFTSYWMHWVKQRPGQGLEWIGAIYPGNSDTSYNQKFKGKAKLTAVTSASTAYMELSSLTHEDSAVYYCTRRGYYYGSSSHFDYWGQGTTLTVSSGGGGSGGGGSGGGGSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPTFGGGTKLEIK

SEQ ID NO: 24 - DNA, 2196 bp, Artificial Sequence
  Synthetic polynucleotide
gaagtgaagcttgaggagtctggaggaggcttggtgcaacctggaggatccatgaaactctcctgtgttgcctctggattcactttcagtaactactggatgaactgggtccgccagtctccagagaaggggcttgagtgggttgctcaaattagattgagatctgatgattatgcaacacattatgcggagtctgtgaaagggaggttcaccatctcaagagatgattcccaaagtagtgtctacctgcaaatgaacaacttaagggctgaagacactggaatttattactgcacaggccccgctaactggggttactggtacttcgatgtctggggcacagggaccacggtcaccgtctcctcaggtggcggaggatctggcggaggcggtagtggcggtggcggatctgatgttttgatgacccaaactccactctccctgcctgtcagtcttggagatcaagcctccatctcttgcagatctagtcagagcattgtacagactaatggaaacacctatttagaatggtacctgcagaaaccaggccagtctccaaagctcctgatctacaaagtttccaaccgattttctggggtcccagacaggttcagtggcagtggatcagggacagatttcacactcaagatcagtagagtggaggctgaggatctgggagtttattactgctttcaaggttcacatgttccgctcacgttcggtgctgggaccaagctggagctgaaaggcggtggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggcggaggtgaggttcagctccagcagtctgggactgtgctggcaaggcctggggcttcagtgaagatgtcctgcaagacttctggctacacatttaccagctactggatgcactgggtaaaacagaggcctggacagggtctggaatggataggggctatttatcctggaaatagtgatactagctacaaccagaagttcaagggcaaggccaaactgactgcagtcacatccgccagcactgcctacatggagctcagcagcctgacacatgaggactctgcggtctattactgtacaagaaggggctattactacggtagtagctcccactttgactactggggccaaggcaccactctcacagtctcctcaggtggcggaggatctggcggaggcggtagtggcggtggcggatctgatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctccgacgttcggtggaggcaccaagctggaaatcaaatga

SEQ ID NO: 25 - DNA, 758 bp, Artificial Sequence
  Synthetic polynucleotide
actagtgttcatcggagcccaggtttactcccttaagtggaaatttcttcccccactccctccttggctttctccaaggagggaacccaggctactggaaagtccggctggggcggggactgtgggtttcagggtagaactgcgtgtggaacgggacagggagcggttagaagggtggggctattccgggaagtggtggggggagggagcccaaaactagcacctagtccactcattatccagccctcttatttctcggccccgctctgcttcagtggacccggggagggcggggaagtggagtgggagacctaggggtgggcttcccgaccttgctgtacaggacctcgacctagctggctttgttccccatccccacgttagttgttgccctgaggctaaaactagagcccaggggccccaagttccagactgcccctcccccctcccccggagccagggagtggttggtgaaagggggaggccagctggagaacaaacgggtagtcagggggttgagcgattagagcccttgtaccctacccaggaatggttggggaggaggaggaagaggtaggaggtaggggagggggcggggttttgtcacctgtcacctgctccggctgtgcctagggcgggcgggcggggagtggggggaccggtataaagcggtaggcgcctgtgcccgctccacctctcaagcagccagcgcctgcctgaatctgttctgccccctccccacccatttcaccaccacc

SEQ ID NO: 26 - DNA, 2393 bp, Artificial Sequence
  Synthetic polynucleotide
ctagtactatgctgcacaagcaatttaaaacaccaacagcaaaaaaatacacttctctgaaaaagtcttggtctaggacctaaacaattgcctgaaactgggtagacttacaccaatgagaggcagataaagagattaagattgagggagtagggcagggcttgcaatggtgccggccaggatgtggctgagggggtgtgggtgcctgccgtggatgctagggtagaagacgactctattaactgggtggctgtaagcagtacccaggtcaatgcctttcatcttctacaacctcgacgttgcctggaatcctaaatctttttcttcacttaacaaacatcacctctgctcaaatctgcaactgctttgatatcacactgcctttttcacccctctattatagatggcatttatttacttacatgttttttccccactagactatactccttgagaacagcgattgtgtcttatttatttctgaatcaccaattcagacaggcatgcaaacacttgttgaaccaatgcacaaatatattttgctcttcttcatagattcctccggcctcagatgaccaggcaccactagatacagaacactgtgctttccttctccaaggtaaaggaataaatatctgttccccttcatgaagtgttactgttgggcctttatgccatcctgaagccaccaggatgtggaaccagatcagggaggtccacagttacaaccccttgtatctgtaacaccagcaggacattatctacagagtcctgctgcagggccccgaatgaagacagcattttgctgctttgtagcgtgagcagtgctgtaacagtgatgcatggatgttcctctggtgtcctgaaagaatgtaggtgcttcttgaaagctctctgcaacttattaattgggagtgattatgcgatggagaaaacagagtcccatcaccccctcagtcttccctgggaaatcacaagagggctgatagctctctgtgaggtgaaccgtttctagaatccccaccgtctcgtcctgttcttccgcccacccagttcctcaagatagcccctgtgggcttctgatgaagtcaccacaccactggctaatgaagtagataaaccagaacagtttggtttaacatttaaggtcagaaacaggaactttctagaggagaaatcaaaaaagcaaaagaagtataagggcagccctccaaccagtcagaataccgtgaccacctgagaggcccgtggcccagcggacacggacgcatgtcaactctggagcagatatcttcagcgcagcatctgacctgggagtacagccacataccctcattcctaaacggcagattgactactgcagccacacacagtctccgggcaatgtggagacatgtctaatatttagtcaacataactcagggtgccacagtcttcacaactgttgtgagcacttgaggatgctgcatttgaagataggaatttgccctcaagcatctggggtttgggtacagaacagagcttcccctgccaccacctgctaattttataaaatgtgcattcaaaaaaaaatcctgcctgtaagaaggaattaagctacccatttaaatataacagctgcctgtgcaatctactgctgctctttataggaaacgcttaaataattgagatacttaattgggttaaagagatccctagcacatagatgttctataaataaaagaatgagtaaataatctagtaaccttccttttcatgtccttcacttaaagagatcgttctgttttgtttgcaccaataagatcactgttagaggactccagagaggtttgatttcaggtggggtggggctttcccaaggaagtcccttttcatttgtttcaggtgtactgccacctttttccctggctctttcactaaaaatgaaaaatttgttgatctttgctgtaagtaggtaggcatctgggctttgcttttgcaactagagtcaaagaagtcaagttatcaggctgatcttgccttgctatctagaatcagaaaggtttaagtagcccagggactactcaaagacagctggaggagaaagggagagagaaaaatgcttataaagaggtgggcaaaagagcgggaccttgtctcaaaaaaaaaaaaaaaaaagaggaagtggtaggaggtgtctgaatttcactgtgacctgttctgtcaggtgatttttggtggggcggggacatgaaaaaaaagttaaaatgtccttataaagacaaaatctttttctttcctggctgatgatttgtcattctagtcacttcctgccttgtgaccacacacccaggcttgacaaagctgttctgcagatcagaaagaaggggttcctggtcatacaccagtactac

SEQ ID NO: 27 - DNA, 428 bp, Artificial Sequence
  Synthetic polynucleotide
gccctggagagcatggggagacccgggaccctgctgggtttctctgtcacaaaggaaaataatccccctggtgtgacagacccaaggacagaacacagcagaggtcagcactggggaagacaggttgtcctcccaggggatgggggtccatccaccttgccgaaaagatttgtctgaggaactgaaaatagaagggaaaaaagaggagggacaaaagaggcagaaatgagaggggaggggacagaggacacctgaataaagaccacacccatgacccacgtgatgctgagaagtactcctgccctaggaagagactcagggcagagggaggaaggacagcagaccagacagtcacagcagccttgacaaaacgttcctggaactcaagctcttctccacagaggaggacagagcagacagcagagacc

SEQ ID NO: 28 - DNA, 1567 bp, Artificial Sequence
  Synthetic polynucleotide
ttgaggtacctggtgtagttttatttcaggttttatgctgtcattttcctgtaatgctaaggacttaggacataactgaattttctattttccacttcttttctggtgtgtgtgtatatatatatgtatatatacacacacacatgtacatatatatattttttagtatctcaccctcacatgctcctccctgagcactacccatgatagatgttaaacaaaagcaaagatgaaattccaactgtcaaaatctcccttccatctaattaattcctcatccaactatgttccaaaacgagaatagaaaattagccccaataagcccaggcaactgaaaagtaaatgctatgttgtactttgatccatggtcacaactcataatcttggaaaagtggacagaaaagacaaaagagtgaactttaaaactcgaatttattttaccagtatctcctatgaagggctagtaaccaaaataatccacgcatcagggagagaaatgccttaaggcatacgttttggacatttagcgtccctgcaaattctggccatcgccgcttcctttgtccatcagaaggcaggaaactttatattggtgacccgtggagctcacattaactatttacagggtaactgcttaggaccagtattatgaggagaatttacctttcccccctctctttccaagaaacaaggagggggtgaaggtacggagaacagtatttcttctgttgaaagcaacttagctacaaagataaattacagctatgtacactgaaggtagctatttcattccacaaaataagagttttttaaaaagctatgtatgtatgtgctgcatatagagcagatatacagcctattaagcgtcgtcactaaaacataaaacatgtcagcctttcttaaccttactcgccccagtctgtcccgacgtgacttcctcgaccctctaaagacgtacagaccagacacggcggcggcggcgggagaggggattccctgcgcccccggacctcagggccgctcagattcctggagaggaagccaagtgtccttctgccctcccccggtatcccatccaaggcgatcagtccagaactggctctcggaagcgctcgggcaaagactgcgaagaagaaaagacatctggcggaaacctgtgcgcctggggcggtggaactcggggaggagagggagggatcagacaggagagtggggactaccccctctgctcccaaattggggcagcttcctgggtttccgattttctcatttccgtgggtaaaaaaccctgcccccaccgggcttacgcaatttttttaaggggagaggagggaaaaatttgtggggggtacgaaaaggcggaaagaaacagtcatttcgtcacatgggcttggttttcagtcttataaaaaggaaggttctctcggttagcgaccaattgtcatacgacttgcagtgagcgtcaggagcacgtccaggaactcctcagcagcgcctccttcagctccacagccagacgccctcagacagcaaagcctacccccgcgccgcgccctgcccgccgctg

SEQ ID NO: 29 - DNA, 266 bp, Artificial Sequence
  Synthetic polynucleotide
gttctttgaaagcagtcgagggggtgctaggtgtgggcagggacgagctggcgcggcgtcgctgggtgcaccgcgaccacgggcagagccacgcggcgggaggactacaactcccggcacaccccgcgccgccccgcctctactcccagaaggccgcggggggtggaccgcctaagagggcgtgcgctcccgacatgccccgcggcgcgccattaaccgccagatttgaatcgccggacccgttggcagaggtggcggcggcggca

SEQ ID NO: 30 - DNA, 60 bp, Artificial Sequence
  Synthetic polynucleotide
tccttcggctgcgggccggctcggctacgcgctctgctccgagccgctcactgcatggta

SEQ ID NO: 31 - DNA, 60 bp, Artificial Sequence
  Synthetic polynucleotide
gggaccagaagggggtgtggcctctcaggtcgaggcggggttaagggtcataaggcggag

SEQ ID NO: 32 - protein, 19 aa, Artificial Sequence
  Synthetic polypeptide
MEWSWVFLFFLSVTTGVHS

SEQ ID NO: 33 - protein, 17 aa, Artificial Sequence
  Synthetic polypeptide
KLHHRCLQALCCRKKPG

SEQ ID NO: 34 - protein, 14 aa, Artificial Sequence
  Synthetic polypeptide
HRCLQALCCRKKPG

SEQ ID NO: 35 - protein, 14 aa, Artificial Sequence
  Synthetic polypeptide
HRCLEELCCRKKPG

SEQ ID NO: 36 - protein, 14 aa, Artificial Sequence
  Synthetic polypeptide
HRCLQQLCCRKKPG

SEQ ID NO: 37 - protein, 595 aa, Artificial seuqence
  Synthetic Polypeptide
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVYEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 38 - protein, 123 aa, Artificial Sequence
  Synthetic Polypeptide
KSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNFKEYKCKVSNKALPAPIEKTISKAK

SEQ ID NO: 39 - protein, 255 aa, Homo Sapiens
MKKTAIAIAVALAGFATVAQADVESKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0353901 A1
(43) Pub. Date: Nov. 20, 2025

(54) FUSION PROTEINS AGAINST SIALOSYLATED GLYCOSPHINGOLIPIDS AND SIALATED GLYCOPROTEINS AND USES THEREOF

(71) Applicant: On. Target Molecules Biotech Inc., Atlanta, GA (US)

(72) Inventor: Francis V A Fernandes, Wilmington, NC (US)

(21) Appl. No.: 17/633,473

(22) PCT Filed: Aug. 7, 2020
(86) PCT No.: PCT/US20/45481

§ 371 Date: Feb. 7, 2022

Related U.S. Application Data
(60) Provisional application No. 62/884,585, filed on Aug. 8, 2019.

(51) Int. Cl. C07K 16/18; A61P 35/00; C12N 15/86

(52) U.S. Cl. CPC C07K 16/18; A61P 35/00; C12N 15/86; C07K 2317/24; C07K 2317/52; C07K 2317/565; C07K 2319/02; C12N 2710/10243

(57) ABSTRACT

The present invention relates to the fusion proteins against a glycocalyx, found to be associated with several human post-translational modified proteins linked to cancer cell lines. The fusion proteins of the present invention are able to bind only to sialylated glycosphingolipids and sialylated glycoproteins and well as their independent constituents the monosaccharide sugars such as neu5ac, galnac and gal that constitute the glycocalyx. The products claimed can be used for diagnosis and treatment of various cancers. Apoptosis via Caspase 3 occurs in cancer cells when the fusion protein bound to targets is sequestered in lysosomes. The fusion protein is not seen in any other organelle of the cell except for lysosomes.

### CROSS REFERENCE TO RELATED APPLICATIONS

This application is a U.S. national stage of International Application No. PCT/US2020/045481, filed Aug. 7, 2020, which claims the benefit of U.S. Provisional Patent Application No. 62/884,585 filed Aug. 8, 2019, the entire contents of each of which are fully incorporated herein by reference.

### INCORPORATION BY REFERENCE OF MATERIAL SUBMITTED ELECTRONICALLY

A Sequence Listing, which is a part of the present disclosure, is submitted concurrently with the specification as a text file. The name of the text file containing the Sequence Listing is “54632A_Seqlisting.txt.” The Sequence Listing was created on Feb. 7, 2022, and is 39,839 bytes in size. The subject matter of the Sequence Listing is incorporated herein in its entirety by reference.

### TECHNICAL FIELD OF THE INVENTION

The present invention relates to the fusion proteins against a glycocalyx, found to be associated with several human post-translational modified proteins linked to cancer cell lines. The fusion proteins of the present invention are able to bind to sialylated glycosphingolipids and sialylated glycoproteins as well as their independent constituents, the monosaccharide sugars such as neu5ac, galnac and gal that constitute the glycocalyx. The products claimed can be used for diagnosis and treatment of various cancers. Apoptosis via Caspase 3 across several cancer cell lines occurs when the fusion protein treatment of cancer cell lines bind targets which are sequestered in lysosomes in seconds. Fusion protein is not observed in any other part of the cell.

### BACKGROUND OF THE INVENTION

The glycocalyx of the invention relates to the carbohydrate moieties in combination with the glycolipids and glycoproteins found on the surface of mammalian cells. Glycolipids include the key group called glycosphingolipids. Glycoproteins are either N- or O-glycosylated. Glycosphingolipids (GSL) and glycoproteins (GC) are responsible for some of the manifold functions of biological membranes.

Glycosphingolipids are composed of three basic structural units: a base, a fatty acid, and a carbohydrate. The lipid moiety of GSL contains a long chain amino-alcohol, the most common being sphingosine, to which a fatty acid is linked via an amide bond. This structure is called ceramide. The hydrophilic carbohydrate unit is linked to the primary hydroxyl group of sphingosine by a glycosidic bond. The carbohydrate is a mono- or usually an oligosaccharide composed of D-glucose, D-galactose, D-mannose, L-fucose, N-acetyl-D-glucosamine, N-acetyl-D-galactosamine, and/or N-acetylneuraminic (sialic) acid. Such carbohydrate residues are often found also as components of membrane glycoproteins (GCs).

Glycosphingolipids are usually located in the outer leaflet of the plasma membrane. Several functions have been ascribed to them: They confer structural rigidity to membranes, are involved in ion transport through membranes, display receptor functions towards glycoprotein hormones, lymphokines, bacterial toxins and the like, and are cell surface antigens and markers involved in cell growth and cell interaction. The latter property has been studied in connection with tumourigenesis and metastasis. Alterations of GSL composition are associated with malignancy, and a few unique GSL antigens are found only in tumours.

Antibodies have been raised against tumour cell surface structures including these antigenic glycosphingolipids with the prospect of gaining valuable tools in tumour diagnosis and immunotherapeutics. Hakomori (Bulletin du Cancer, Paris, 70, 118 (1983)) reviews the glycolipid changes associated with oncogenic transformations and the use of monoclonal antibodies in this context.

Due to the relationship of the carbohydrate residue of glycosphingolipids and membrane glycoproteins, antibodies raised against glycolipids may recognize also glycoproteins and vice versa.

GLS are a class of lipids containing a backbone of sphingoid bases, a set of aliphatic amino alcohols that includes sphingosine. These compounds play an important role in cell structure of the membrane but in the case of complex sphingolipids it is recently been found that they play an important role in signal transduction, cell recognition and in immunology. A simple sphingolipid with a fatty acid and the terminal hydroxy group is a ceramide. The terminal hydroxy group of the ceramide can be substituted with a number of groups in mammalian cells to create the complex shingolipids these include; phosphocholine or phosphoethanolamine, yielding a sphingomyelin, various sugar monomers or dimers or oligosaccharides, yielding cerebrosides (one sugar) and globosides (more than one sugar), respectively. Cerebrosides and globosides are collectively known as glycosphingolipids. Where more than one sugar is present it is normally a mixture of sugars with the bonding between the rings designated by numbers with the following type of nomenclature, for example

$$GM 3 = Neu 5 Ac α 2 - 3 Gal β 1 - 4 Glcp β 1 - 1 Cer$$

(wherein Cer is ceramide;
Neu5Acαis 5-acetyl-alpha-neuraminic acid [often called NANA];
Neu5Ac9Acαis 5,9-diacetyl-alpha-neuraminic acid;
Galβis beta-D-galactopyranose;
GalβNAc is N-acetyl-beta-D-galactopyranose; and
Glcβbeta-D-glucopyranose).

The sialic acid (sialyl) family includes 43 derivatives of the nine-carbon sugar neuraminic acid—such as “Neu5Acα” which is 5-acetyl-alpha-neuraminic acid; “Neu5Ac9Acα” which is 5,9-diacetyl-alpha-neuraminic acid, as explained above.

When the globoside is formed with an oligosaccharide it may also have one or more sialyl groups (sialylation) and this is called a ganglioside. Gangliosides are also classified as a glycosphingolipids and more than 60 types have been classified in humans the variation occurring in the oligosaccharide chain, as outlined above, and the number of sialyl groups. Additionally, it has been found that cancerous cells can produce gangliosides on the cell surface not found in healthy cells.

Additionally, it has been found that the sphingolipid metabolite, ceramide, has recently emerged as an important second messenger that may mediate a number of biological processes, including induction of cell-death. Ceramide is produced in vivo by de novo biosynthesis and by turnover of complex sphingolipids. In the latter pathway, ceramide is produced by activation of sphingomyelinases in response to a variety of apoptotic agonists including the tumor necrosis factor-a (TNFa). De novo synthesis of ceramide occurs at the cytosolic face of the endoplasmic reticulumand is initiated by the condensation of serine and palmitoyl-CoA catalyzed by serine palmitoyltransferase. Various antitumor agents induce apoptosis through de novo biosynthesis of ceramide. In order to mediate its cellular effects, ceramide has been shown to activate a number of enzymes involved in stress signaling cascades including protein kinases, protein phosphatases and caspases as well as mitochondrial alterations.

Changes in glycosylation of glycosphingolipids are associated with the activity of transferase enzymes regulated by oncogenes. Sialylation in particular is associated with carcinogenesis, metastasis and a poor prognosis for cancer patients. The expression level of sialyl transferases has been utilized as a prognostic marker for staging several cancer types. The paratope map of transferase enzymes indicates amino acids that bind the sugar motifs of the glycocalyx, such as Galβ, GalβNAc andNeu5Acα.

The human P2X7 receptor gene has been shown to have many single nucleotide polymorphisms (SNPs) including ones that lead to loss of function of the receptor. P2X7 protein is a 595 amino acid protein with a predicted structure comprising two transmembrane domains and a bulky extracellular cysteine rich region, with conserved lysine and glycine residues and several potential N-linked glycosylation sites, followed by a long stretch forming six putative antiparallel β-sheets. The amino acid and carboxyl-terminal domains are both cytoplasmic.

P2X7 is an ionotropic, ligand-gated, cation channel. Stimulation of the receptor with low ATP doses reversibly opens a membrane channel permeable to small cations, while sustained stimulation with higher ATP doses or repeated stimulation with sequential ATP pulses, induces the formation of a pore permeable to large molecular weight molecules.

The carboxylic-terminal cytoplasmic domains of P2X7 receptor comprise amino acids 352 to 595 and are longer than in other members of the P2X subtype. This domain is crucial for P2X7 pore formation, transduction and signaling. Allelic mutations, leading to loss of function, have been identified in the human and mouse receptor. It has been suggested that pore formation requires over 95% of the C-terminal tail of the receptor.

The glutamic acid 496 seems to be important for the pore-forming activity of the P2X7 receptor and substitution of glutamic acid (Glu) with alanine (Ala) (E496A), occurring in the ankyrin repeat motif of the carboxyl-terminal domain of the receptor that leads to loss of function of the receptor in homozygous individuals and around 50% reduction in heterozygous individuals (Adinolfi et al., Purinergic Signaling, 2005, 1:219-227).

In general mutagenesis may not lead to carcinogenesis. However, the presence of a particular glycocalyx seems to be indicative of numerous cancers. Hence, there exists a need for recognizing post translational modifications that would discriminate between the glycocalyx of mammalian receptor proteins that are indicative of cancerous cells and the gylycocalyx of mammalian receptors cells that are not indicative of being cancerous cells. The differences in the glyocalyx that distinguish between the gylcocalyx of the cancerous and non-cancerous cell lines are preferably Neu5Acα2-3Galβ1-3GalNAcα-R and Neu5Acα2-6GalNAc α-R which are a known biological markers for cancer—wherein R is the protein to which it is attached.

According to the present invention binding molecules that demonstrate binding selectivity between the glycocalyx of cancerous and non-cancerous mammalian cells. These molecules were raised against the antigen KLH-HRCLQALCCRKKPG. This sequence HRCLQALCCRKKPG includes the SNP mutation to A from E at position 496 of P2X7 and an additional amino acid replacement E to Q at position 495 (E495Q) and it is conjugated to Keyhole limpet hemocyanin (KLH).

Therefore, we present as a feature of the invention a binding molecule for KLH-HRCLQALCCRKKPG and methods for preparing such a binding molecule as described herein.

### SUMMARY OF THE INVENTION

The inventors have surprisingly demonstrated that a fusion protein according to the invention leads to apoptosis in breast cancer, breast ductal cancer, triple-negative cancer, lung carcinoma, small lung cell carcinoma, B-cell leukaemia, prostate carcinoma, melanoma, bladder cancer, colon cancer, glioblastoma, liver cancer, prostate cancer, cervical cancer, ovarian cancer, head and neck cancer and bladder cancer cell lines whilst having no apoptotic effect in a non-cancerous cell lines.

As described above, the effects observed with the fusion protein are observed in a wide array of different cancer cell lines, which indicates that the fusion protein according to the invention can be used as a general treatment for all cancers. Moreover, the fusion protein is selective for cancer cells because it does not cause cell death in healthy cells and therefore it is expected that the fusion protein will have few side effects.

Changes in glycosylation of glycosphingolipids are associated with the activity of transferase enzymes regulated by oncogenes. Sialylation in particular is associated with carcinogenesis, metastasis and a poor prognosis for cancer patients. The expression level of sialyl transferases has been utilized as a prognostic marker for staging several cancer types. The paratope map of transferase enzymes indicates amino acids that bind the sugar motifs of the glycocalyx, such as Gal, GalβNAc and Neu5Acα

Fusion proteins of the invention have been generated that exhibit a high selectivity for the glycocalyx specific for cancer cells shown as follows:

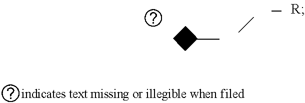

wherein R is a peptide sequence having a site capable of glycosylation with an O-glycan. In one example the peptide sequence is glycosylated with an O-glycan. In a further example, the peptide can contain a serine and/or threonine with an O-glycan glycosylation. A peptide sequence having a site capable of glycosylation with an O-glycan can be identified by techniques know in the art. Non-limiting examples of suitable techniques include protein painting and heavy isotope techniques.

Further evidence for this glycan biomarker target comes from mass spectroscopy data from one bladder cancer cell line and one prostate cancer cell line, where the enzyme ST3GAL1 is downregulated on treatment of these cell lines in-vitro with the fusion protein. The sialyltransferase add sialic acid to galactose of the core Gal(ß1-3)GalNAc-determinant of o-glycans and glycosphingolipids such as GM2. More evidence is also present from data mining where Serine/Threonine glycosylation sites are shown as positives.

As used herein the term “O-glycosylation” relates to the transfer of GalNAc to serine and threonine residues on proteins by a family of UDP-GalNAc:polypeptide N-acetylgalactosaminlytransferases.

As used herein the term “Sialylation” relates to the process by which sialic acid groups are introduced as the terminal monosaccharide molecules onto oligosaccharides and carbohydrates. Sialic acid is a general term for N or O substituted derivatives of neuraminic acid which are widely expressed terminal carbohydrates on cell surface glycoproteins and glycolipids of eukaryotic cells.

Accordingly, the invention provides a fusion protein comprising:
(i) a first binding domain comprising:
   (a) a first variable heavy domain (V_{H1}) having a sequence comprising:
      (i) a vhCDR1 having at least 90% identity to SEQ ID NO: 4;
      (ii) a vhCDR2 having at least 90% identity to SEQ ID NO: 5; and
      (iii) a vhCDR3 having at least 90% identity to SEQ ID NO: 6; and
   (b) a first variable light domain (V_{L1}) having a sequence comprising:
      (i) a vlCDR1 having at least 90% identity to SEQ ID NO: 7;
      (ii) a vlCDR2 having at least 90% identity to SEQ ID NO: 8; and
      (iii) a vlCDR3 having at least 90% identity to SEQ ID NO: 9; and
(ii) a second binding domain comprising:
   (a) a second variable heavy domain (V_{H2}) having a sequence comprising:
      (i) a vhCDR1 having at least 90% identity to SEQ ID NO: 10;
      (ii) a vhCDR2 having at least 90% identity to SEQ ID NO: 11; and
      (iii) a vhCDR3 having at least 90% identity to SEQ ID NO: 12; and
   (b) a second variable light domain (V_{L2}) comprising:
      (i) a vlCDR1 comprising at least 90% identity to SEQ ID NO: 13;
      (ii) a vlCDR2 comprising at least 90% identity to SEQ ID NO: 14; and
      (iii) a vlCDR3 comprising at least 90% identity to SEQ ID NO: 15; and
   (iii) a human Fc domain (Fc);
   wherein V_{H1} and V_{L1} are connected by a first linker (L₁) and V_{H2} and V_{L2} are connected by a second linker (L₂).

In one embodiment the first binding domain comprises:
(a) a first variable heavy domain (V_{H1}) having a sequence comprising:
   (i) a vhCDR1 consisting of SEQ ID NO: 4;
   (ii) a vhCDR2 consisting of SEQ ID NO: 5; and
   (iii) a vhCDR3 consisting of SEQ ID NO: 6; and
(b) a first variable light domain (V_{L1}) having a sequence comprising:
   (i) a vlCDR1 consisting of SEQ ID NO: 7;
   (ii) a vlCDR2 consisting of SEQ ID NO: 8; and
   (iii) a vlCDR3 consisting of SEQ ID NO: 9; and
and wherein the second binding domain comprises:
(a) a second variable heavy domain (V_{H2}) having a sequence comprising:
   (i) a vhCDR1 consisting of SEQ ID NO: 10;
   (ii) a vhCDR2 consisting of SEQ ID NO: 11; and
   (iii) a vhCDR3 consisting of SEQ ID NO: 12; and
(b) a second variable light domain (V_{L2}) comprising:
   (i) a vlCDR1 consisting of SEQ ID NO: 13;
   (ii) a vlCDR2 consisting of SEQ ID NO: 14; and
   (iii) a vlCDR3 consisting of SEQ ID NO; 15.

In one embodiment:
(a) the V_{H1} comprises a sequence having at least 90% identity to SEQ ID NO: 16;
(b) the V_{L1} comprises a sequence having at least 90% identity to SEQ ID NO: 17;
(c) the V_{H2} comprises a sequence having at least 90% identity to SEQ ID NO: 18; and
(d) the V_{L2} comprises a sequence having at least 90% identity to SEQ ID NO: 19.

In one embodiment:
(a) the V_{H1} consists of SEQ ID NO: 16;
(b) the V_{L1} consists of SEQ ID NO: 17;
(c) the V_{H2} consists of SEQ ID NO: 18; and
(d) the V_{L2} consists of SEQ ID NO: 19.

In one embodiment the fusion protein further comprises a signal peptide (S), preferably wherein the signal peptide (S) is located upstream of the amino terminus of the fusion protein, even more preferably wherein the signal peptide is upstream of the amino terminus of V_{H1}.

In one embodiment the amino acid sequence of L₁ and L₂ are identical; optionally wherein L₁ is located downstream of the carboxy terminus of V_{H1} and upstream of the amino terminus of V_{L1} and/or wherein L₂ is located downstream of the carboxy terminus of V_{H2} and upstream of the amino terminus of V_{L2}. Preferably, each of L₁ and L₂ have a sequence that has at least 90% identity to SEQ ID NO: 20. Alternatively, each of L₁ and L₂ have a sequence that consists of SEQ ID NO: 20.

In one embodiment the peptide further comprises a third linker (L₃) and a fourth linker (L₄); and wherein L₃ is located downstream of the carboxy terminus of V_{L1} and upstream of the amino terminus of the Fc and L₄ is located downstream of the carboxy terminus of the Fc and upstream of the amino terminus of V_{H2}. Preferably, each of L₃ and L₃ have a sequence that has at least 90% identity to SEQ ID NO: 21. Alternatively, each of L₃ and L₃ have a sequence that consists of SEQ ID NO: 21

In one embodiment the human Fc domain is selected from: IgG, IgE, IgM and IgA. Preferably the human Fc domain is selected from: IgG1, IgG2, IgG3, and IgG4. The human Fc domain may a sequence that is at least 90% identity to SEQ ID NO: 22. In one embodiment the human Fc domain has a sequence that consists of SEQ ID NO: 22.

In one embodiment the fusion protein is arranged from amino-terminus to carboxy-terminus in an arrangement selected from:

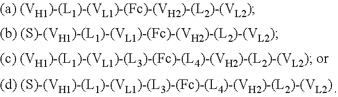

In one embodiment the protein has a sequence that is at least 90% identical to SEQ ID NO: 23.

In one embodiment the peptide has a sequence that consists of SEQ ID NO: 23.

In another aspect, the invention provides a fusion protein comprising:
(a) a first binding domain;
(b) a second binding domain; and
(c) a human Fc domain (Fc);
wherein each of the first and second binding domains selectively bind to Neu5Acα2-3Galβ1-3GalNAcα-R.

In another aspect, the invention provides a nucleic acid sequence encoding the fusion protein as claimed in any preceding claim.

In another aspect, the invention provides an expression cassette comprising a promoter operably linked to the nucleic acid according to the invention. the promoter may be selected from:
(i) SEQ ID NO: 25:
(ii) SEQ ID NO: 26;
(iii) SEQ ID NO: 27;
(iv) SEQ ID NO: 28;
(v) SEQ ID NO: 29;
(vi) SEQ ID NO: 3;
(vii) SEQ ID NO: 30; and
(viii) SEQ ID NO: 31.

In another aspect, the invention provides an adenoviral vector comprising the expression cassette of the invention.

In one embodiment, the adenoviral vector is:
(a) an oncolytic adenoviral vector; preferably a conditionally replicative adenovirus (CRAd);
(b) a non-replicative adenovirus; preferably a non-replicative Ad5;
(c) a hybrid Ad5/3 adenovirus; or a hybrid Ad5/35 adenovirus.

In another aspect, the invention provides an adenoviral vectors comprising at least one of:
(a) the nucleic acid according to the invention operably linked to SEQ ID NO: 25;
(b) the nucleic acid according to the invention operably linked to SEQ ID NO: 26;
(c) the nucleic acid according to the invention operably linked to SEQ ID NO: 27;
(d) the nucleic acid according to the invention operably linked to SEQ ID NO: 28;
(e) the nucleic acid according to the invention operably linked to SEQ ID NO: 29;
(f) the nucleic acid according to the invention operably linked to SEQ ID NO: 3;
(g) the nucleic acid according to the invention operably linked to SEQ ID NO: 30;
(h) the nucleic acid according to the invention operably linked to SEQ ID NO: 31 and
(i) any combination of (a) to (h) above.

In one embodiment the plurality of adenoviral vectors comprises each of the adenovirus vectors according to clause (a) to clause (h) above.

In another aspect the invention provides a fusion protein according to the invention, or adenoviral vector as according to the invention, for use in medical therapy.

In another aspect the invention provides a fusion protein according to the invention, or adenoviral vector according to the invention, for use in the treatment of cancer; preferably wherein the cancer is selected from: breast, triple-negative breast, melanoma, lung, small cell lung, B-cell leukemia, prostate, bladder, colon, glioblastoma, liver, cervical, ovarian and head and neck cancer.

In another aspect the invention provides a method for treating a disease, wherein the method comprises: administering a therapeutically effective amount of a fusion protein according to the invention, or adenoviral vector according to the invention, to a patient in need thereof.

In another aspect the invention provides a method for treating cancer, wherein the method comprises: administering a therapeutically effective amount of a fusion protein according to the invention, or adenoviral vector according to the invention, to a patient in need thereof.

In one embodiment the cancer is selected from: breast, triple-negative breast, melanoma, lung, small cell lung, B-cell leukemia, prostate, bladder, colon, glioblastoma, liver, cervical, ovarian and head and neck cancer.

In another aspect the invention provides a method for treating cancer, wherein the method comprises: administering a therapeutically effective amount of a fusion protein according to the invention, or fragments or functional variants thereof, or an adenoviral vector according to the invention, to a patient in need thereof; wherein the fusion protein, fragments, functional variants thereof are sequestered to the lysosome of a cancer cell.

In one embodiment the sequestration to the lysosomes occurs in under 15 seconds.

In one embodiment sequestration to the lysosome leads to caspase 3-mediated apoptosis.

In one embodiment sequestration to the lysosome leads to inhibition of sialylation.

In one embodiment the fusion protein is administered at a concentration of between 1 μM and 10 μM.

Other aspects and advantages of the present invention will become apparent to those skilled in the art in view of the following detailed description.

### BRIEF DESCRIPTION OF THE DRAWINGS

Embodiments of the invention are further described hereinafter with reference to the accompanying drawings, in which:

FIG. 1 shows apoptosis measurements in MCF7 cells incubated with the antibodies of example 2. Upper diagram shows relative cell count (%) over different antibody concentrations after 72 hrs incubation. Lower graph shows apoptosis (fold induction) over different antibody concentrations after 72 hours incubation.

FIG. 2 shows apoptosis measurements in MCF7 cells incubated with Staurosporine. Upper diagram shows relative cell count (%) over different Staurosporine concentrations after 72 hrs incubation. Lower graph shows apoptosis (fold induction) over different Staurosporine concentrations after 72 hours incubation.

FIG. 3 shows apoptosis measurements in MCF7 cells incubated with IgG. Upper diagram shows relative cell count (%) over different IgG concentrations after 72 hours incubation. Lower graph shows apoptosis (fold induction) over different IgG concentrations after 72 hours incubation.

FIG. 4 shows an interactive network pathway of apoptosis under HIST control—which is the exact histones received from protein paints.

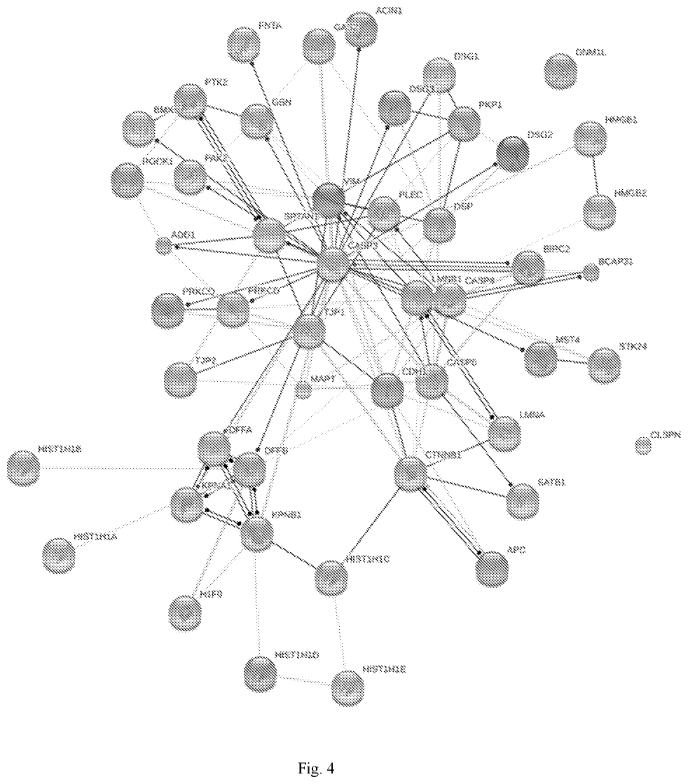

FIG. 5 shows an interactive network pathway of autophagy under ATG4 control in which ATG4B upregulated.

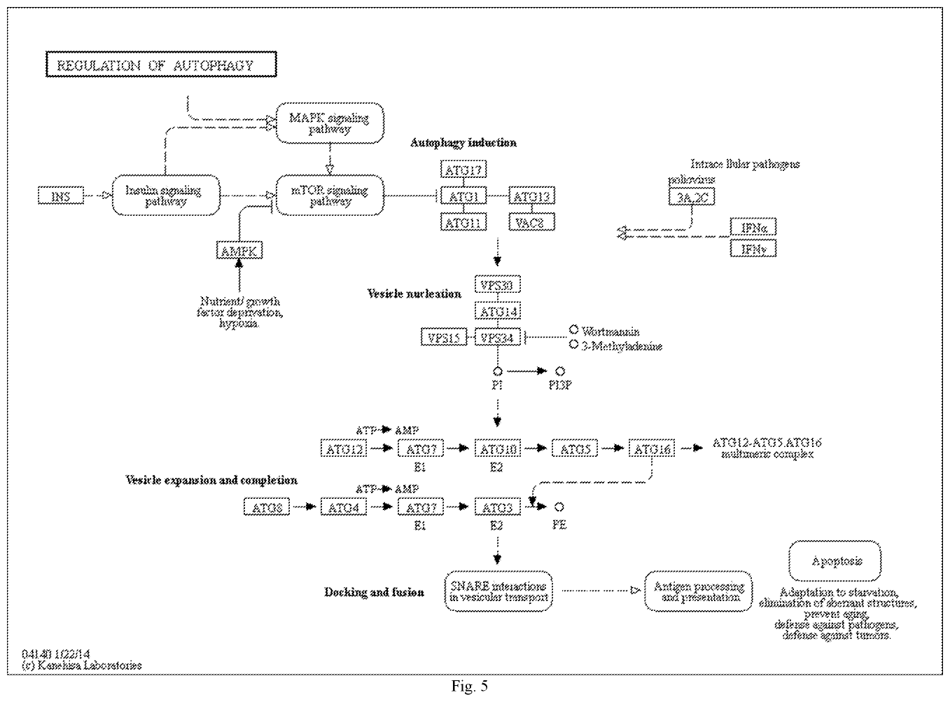

FIG. 6 shows relative cell death by apoptosis and autophagy in vitro.

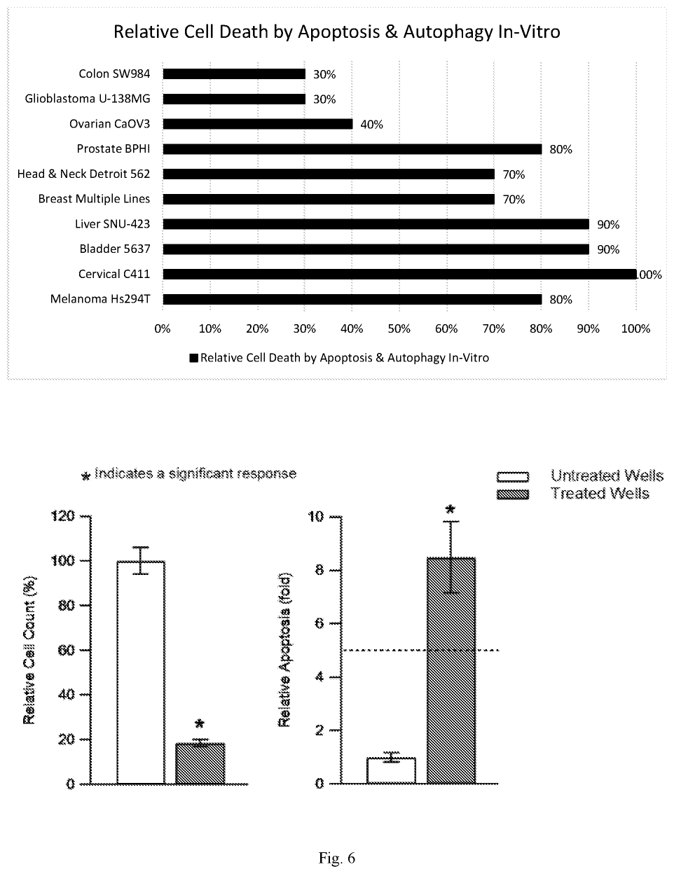

FIG. 7 shows cell apoptosis assay results for different cell lines. The cell lines were dosed on day 1 and day 4 with the fusion protein and cell death was measured on day 8. Top panel: Results from A549 lung carcinoma cell line; middle panel BT474 breast ductal carcinoma cell line; bottom panel DMS53 small cell lung carcinoma cell line. Top panel MDA-MB-231 triple negative breast cancer cell line; bottom panel MCF7 breast cancer cell line.

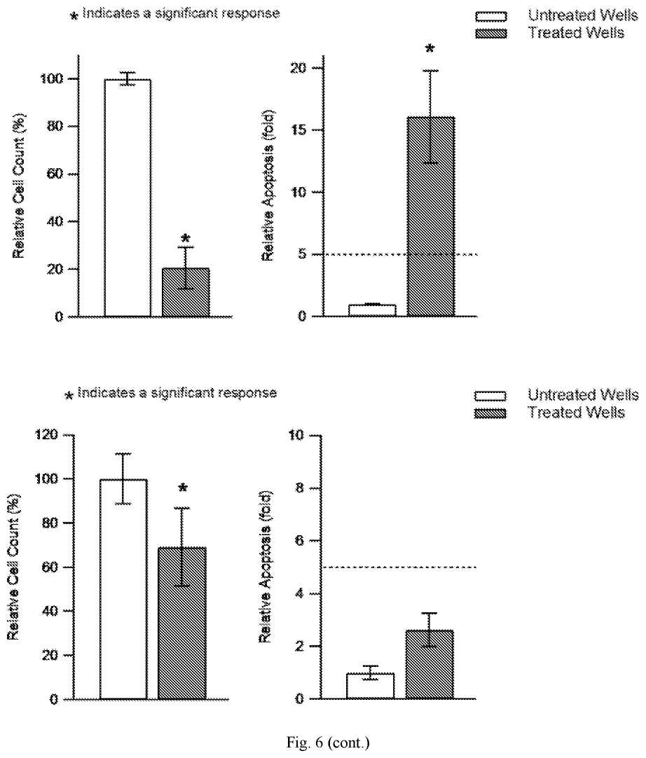

FIG. 8 shows that there was no cell apoptosis when the fusion protein was incubated with the normal breast cell line MCF10A.

FIG. 9 shows that there was no cell apoptosis when the fusion protein was incubated with the normal breast cell line MCF10A.

FIG. 10 shows that there was apoptosis when staurosporine was incubated with the normal breast cell line MCF10A.

FIG. 11 shows microscopy of hepatocytes in which no apoptosis was observed at 48 hours after fusion protein treatment at the therapeutic dose in-vitro.

FIG. 12 shows zebra fish embryo that was used for toxicity tests over 14 days. Embryos were injected with the fusion protein at twice the concentration of the therapeutic dose on day 1, day 3, day 7, and day 10. Observations were recorded for acute toxicity, hepatoxicity, ototoxicity and cardiotoxicity. Behavioral assays were conducted. In zebra fish embryos, no toxicity was observed over a 14 day investigation.

FIG. 13 shows 45% apoptosis after 72 hours when a cell line of bladder cancer HTB-9 was treated once with Ab (fusion protein?) Blue—living cells Green—apoptosis cells.

FIG. 14 shows fusion protein with 488a dye (green). The lysosomes were stained red. Co-localisation was observed by colour combination where Green+Red=Yellow was observed. Co-localisation was not observed for other organelles such as nucleus, ribosomes, mitochondria, ER, cell membrane.

FIG. 15 shows a confocal microscopy image of the bladder cancer cell line. Green-fusion protein in endosomes-Lysosomes Red-mitochondria.

FIG. 16 shows a confocal microscopy image of the bladder cancer cell line and that the fusion protein is not localized in the mitochondria—red.

FIG. 17 shows a confocal microscopy image of the bladder cancer cell line and that the fusion protein is not localized in the golgi—red.

FIG. 18 shows a confocal microscopy image of triple negative breast cancer cells in which the fusion protein was green fluorophore tagged in the endosomes-lysosomes.

FIG. 19 shows a confocal microscopy image of prostate cancer cell line in which the fusion protein was green fluorophore tagged in the endosomes-lysosomes and not in the mitochondria (red-mitochondria).

FIG. 20 shows the fusion protein was observed in lysosomes in less than 15 seconds in bladder cancer cell line.

FIG. 21 shows that 100% of the fusion protein sequesters in the lysosomes 100%, which contrasts to 3% of a leading brand[Green Ab+Red Lysosome stain=Yellow for co-localization].

FIG. 22 shows a diagrammatic representation of the replication deficient and oncolytic adenovirus vectors generated, wherein the GOI indicates a nucleic acid sequence encoding the fusion protein.

FIG. 23 shows the full length polypeptides sequence of the human P2X7 receptor.

FIG. 24 shows humanization of the IgG1 framework of the binding regions of the invention.

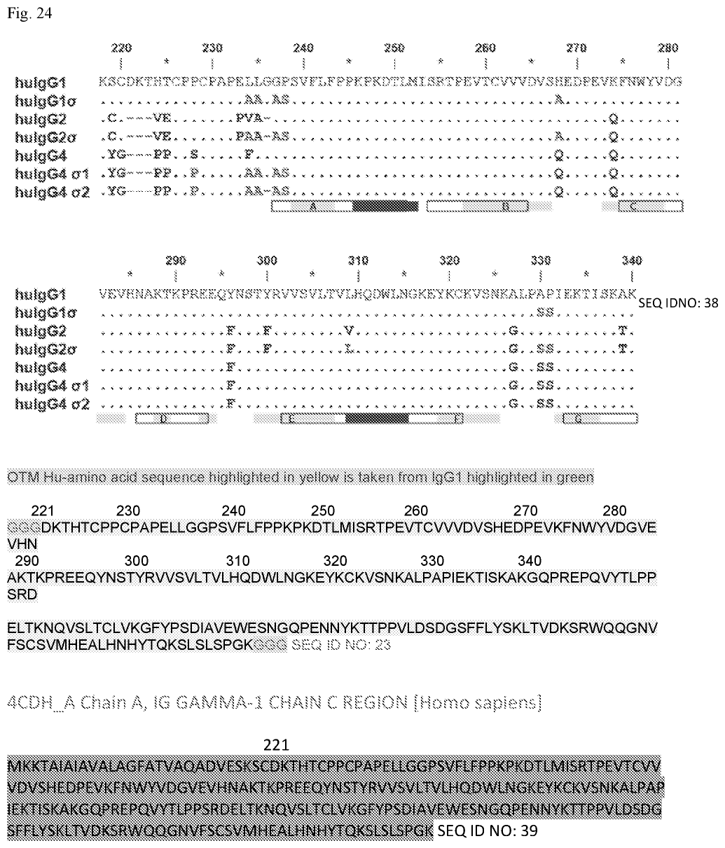

FIG. 25 shows the composition of the Fusion Protein 2G7.A8 scFv-hFc-7F11.B9 scFv.

FIG. 26 shows the mechanism by which autophagy and apoptosis occurs via a Caspase 3 mechanism.

FIG. 27 (A) shows that the green fluorophore tagged fusion protein localizes in lysosomes of triple negative breast cancer cells. The white arrows point to exemplary areas where the fusion protein is located in the lysosomes. (B) shows colocalization of two stains, red stain of lysosomes, and green fluorescent tag on the fusion protein. Here, the colocalization is seen as light grey. Exemplary areas of colocalization are indicated by black arrows.

### DETAILED DESCRIPTION OF THE INVENTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The inventors have surprisingly demonstrated that a fusion protein according to the invention leads to apoptosis in breast cancer, breast ductal cancer, triple-negative cancer, lung carcinoma, small lung cell carcinoma, B-cell leukaemia, prostate carcinoma, melanoma, bladder cancer, colon cancer, glioblastoma, liver cancer, prostate cancer, cervical cancer, ovarian cancer, head and neck cancer and bladder cancer cell lines whilst having no apoptotic effect in a non-cancerous breast cell line.

As described above, the effects observed with the fusion protein are observed in a wide array of different cancer cell lines, which indicates that the fusion protein according to the invention can be used as a general treatment for all cancers. Moreover, the fusion protein is selective for cancer cells because it does not cause cell death in healthy cells and therefore it is expected that the fusion protein will have few side effects.

The present invention provides antibody-derived fusion proteins. The fusion protein according to the present invention comprises a first variable heavy V_{H} domain paired with a first variable light V_{L} domain as well as a second variable heavy V_{H} domain paired with a second variable light V_{L} domain to provide two antibody binding sites comprising both V_{H} and V_{L} domains for two epitopes (i.e. a bispecific fusion protein). The variable domains of the fusion protein are derived from two different antibodies. In the variable regions of antibodies, three loops are gathered for each of the variable domains of the heavy chain and light chain to form the antibody binding site (i.e. antigen-binding site). Each of the loops is referred to as a complementarity-determining region (CDR). The CDRs are the regions of the variable domains in which the amino acid sequence varies considerably between different antibodies. Variability within the variable region is not evenly distributed. Indeed the variable region has a number of framework regions (FRs) of 15-30 amino acids which are highly conserved amongst different antibodies. Each V_{H} and V_{L} is composed of three hypervariable regions (CDRs) and four FRs, arranged from amino-terminus to carboxy-terminus in the following order: FR1-CDR1-FR2-CDR2-FR3-CDR3-FR4. The region of each CDR generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. As will be appreciated by those in the art, the exact numbering and placement of the CDRs can be different among different numbering systems.

However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vlCDRs (e.g. vlCDR1, vlCDR2 and vlCDR3). A useful comparison of CDR numbering can be found in Lafranc et al., Dev. ;Comp. Immunol. 27(1):55-77 (2003). The CDRs contribute to the formation of the antigen-binding, or more specifically, epitope binding site of antibodies.

The terms “antibody” or “antibodies” as used herein refer to molecules or active fragments of molecules that bind to known antigens, particularly to immunoglobulin molecules and to immunologically active portions of immunoglobulin molecules, i.e. molecules that contain a binding site that immunospecifically binds an antigen. The immunoglobulin according to the invention can be of any class (IgG, IgM, IgD, IgE, IgA and IgY) or subclass (e.g. IgG1, IgG2, IgG3, IgG4, IgA1 and IgA2) or subclasses (isotypes) of immunoglobulin molecule (e.g. IgG in IgG1, IgG2, IgG3, and IgG4, or IgA in IgA1 and IgA2).

Within the scope of the present invention the terms “antibody” or “antibodies” include monoclonal, polyclonal, chimeric, single chain, bispecific, human and humanized antibodies as well as active fragments thereof. Examples of active fragments of molecules that bind to known antigens include Fab, F(ab′)2, scFv and Fv fragments, including the products of an Fab immunoglobulin expression library and epitope-binding fragments of any of the antibodies and fragments mentioned above.

As used herein, the term “monoclonal antibody” refers to an antibody that is mass produced in the laboratory from a single clone and that recognizes only one antigen. Monoclonal antibodies are typically made by fusing a normally short-lived, antibody-producing B cell to a fast-growing cell, such as a cancer cell (sometimes referred to as an “immortal” cell). The resulting hybrid cell, or hybridoma, multiplies rapidly, creating a clone that produces large quantities of the antibody. For the purpose of the present invention, “monoclonal antibody” is also to be understood to comprise antibodies that are produced by a mother clone which has not yet reached full monoclonality.

As used herein the term “humanized antibody” or “humanized version of an antibody” refers to antibodies in which the framework or “complementarity determining regions” (CDR) have been modified to comprise the CDR of an immunoglobulin of different specificity as compared to that of the parent immunoglobulin. In some exemplary embodiments, the CDRs of the V_{H} and VL are grafted into the framework region of human antibody to prepare the “humanized antibody.” See e.g. Riechmann, L., et al., Nature 332 (1988) 323-327; and Neuberger, M. S., et al., Nature 314 (1985) 268-270. The heavy and light chain variable framework regions can be derived from the same or different human antibody sequences. The human antibody sequences can be the sequences of naturally occurring human antibodies. Human heavy and light chain variable framework regions are listed e.g. in Lefranc, M.-P., Current Protocols in Immunology (2000)—Appendix 1P A.1P.1-A.1P.37 and are accessible via IMGT, the international ImMunoGeneTics information System® (http://imgt.cines.fr) or via http://vbase.mrc-cpe.cam.ac.uk, for example. Optionally the framework region can be modified by further mutations. Exemplary CDRs correspond to those representing sequences recognizing the antigens noted above for chimeric antibodies. In some embodiments, such humanized version is chimerized with a human constant region. The term “humanized antibody” as used herein also comprises such antibodies which are modified in the constant region to generate the properties according to the disclosure, especially in regard to C1q binding and/or FcR binding, e.g. by “class switching” i.e. change or mutation of Fc parts (e.g. from IgG1 to IgG4 and/or IgG1/IgG4 mutation).

The Kabat numbering system is generally used when referring to a residue in the variable domain (approximately, residues 1-107 of the light chain variable region and residues 1-113 of the heavy chain variable region) and the EU numbering system for Fc regions (e.g, Kabat et al., supra (1991)). The CDRs of the antibodies described herein were identified using VBASE2 (http://vbase2.org/).

Specific CDRs of the fusion protein are described herein. In certain aspects the fusion protein provided herein, comprises:
(a) a first variable heavy domain (V_{H1}) comprising:
   (i) a first CDR comprising at least 90% identity to SEQ ID NO: 4;
   (ii) a second CDR comprising at least 90% identity to SEQ ID NO: 5; and
   (iii) a third CDR comprising at least 90% identity to SEQ ID NO: 6
(b) a first variable light domain (V_{L1}) comprising:
   (i) a first CDR comprising at least 90% identity to SEQ ID NO: 7;
   (ii) a second CDR comprising at least 90% identity to SEQ ID NO: 8; and
   (iii) a third CDR comprising at least 90% identity to SEQ ID NO: 9
(c) a second variable heavy domain (V_{H2}) comprising:
   (i) a first CDR comprising at least 90% identity to SEQ ID NO: 10;
   (ii) a second CDR comprising at least 90% identity to SEQ ID NO: 11; and
   (iii) a third CDR comprising at least 90% identity to SEQ ID NO: 12
(d) a first variable light domain (V_{L2}) comprising:
   (i) a first CDR comprising at least 90% identity to SEQ ID NO: 13;
   (ii) a second CDR comprising at least 90% identity to SEQ ID NO: 14; and
   (iii) a third CDR comprising at least 90% identity to SEQ ID NO: 15
(e) a human Fc domain (Fc);
wherein V_{H1} and V_{L1} are connected by a first linker (L₁) and V_{H2} and V_{L2} are connected by a second linker (L₂).

Each of the CDRs described herein may comprise at least 90%, at least 91%, at least 92%, at least 93%, at least 94%, at least 95%, at least 96%, at least 97%, at least 98% or at least 99% identity to their respective SEQ ID Nos. Alternatively, each of the CDRs described herein may comprise 100% identity to their respective SEQ ID Nos.

In one embodiment the first variable heavy domain (V_{H1}) comprises:
(i) a first CDR having 100% identity to SEQ ID NO: 4;
(ii) a second CDR having 100% identity to SEQ ID NO: 5; and
(iii) a third CDR having 100% identity to SEQ ID NO: 6.

In one embodiment the first variable light domain (V_{L1}) comprises:
(i) a first CDR having 100% identity to SEQ ID NO: 7;
(ii) a second CDR having 100% identity to SEQ ID NO: 8; and
(iii) a third CDR having 100% identity to SEQ ID NO: 9.

In one embodiment the second variable heavy domain (V_{H2}) comprises:
(i) a first CDR having 100% identity to SEQ ID NO: 10;
(ii) a second CDR having 100% identity to SEQ ID NO: 11; and
(iii) a third CDR having 100% identity to SEQ ID NO: 12.

In one embodiment the first variable light domain (V_{L2}) comprises:
(i) a first CDR having 100% identity to SEQ ID NO: 13;
(ii) a second CDR having 100% identity to SEQ ID NO: 14; and
(iii) a third CDR comprising having 100% identity to SEQ ID NO: 15 In one embodiment, the fusion protein comprises:
(a) a first variable heavy domain (V_{H1}) comprising:
   (i) a first CDR having 100% identity to SEQ ID NO: 4;
   (ii) a second CDR having 100% identity to SEQ ID NO: 5; and
   (iii) a third CDR having 100% identity to SEQ ID NO: 6; and optionally or alternatively
(b) a first variable light domain (V_{L1}) comprising:
   (i) a first CDR having 100% identity to SEQ ID NO: 7;
   (ii) a second CDR having 100% identity to SEQ ID NO: 8; and
   (iii) a third CDR having 100% identity to SEQ ID NO: 9, and optionally or alternatively
(c) a second variable heavy domain (V_{H2}) comprising:
   (i) a first CDR having 100% identity to SEQ ID NO: 10;
   (ii) a second CDR having 100% identity to SEQ ID NO: 11; and
   (iii) a third CDR having 100% identity to SEQ ID NO: 12, and optionally or alternatively
(d) a first variable light domain (V_{L2}) comprising:
   (i) a first CDR having 100% identity to SEQ ID NO: 13;
   (ii) a second CDR having 100% identity to SEQ ID NO: 14; and
   (iii) a third CDR having 100% identity to SEQ ID NO: 15.

The fusion protein as described herein may have a V_{H1} comprising at least 90%, at least 91%, at least 92%, at least 93%, at least 94%, at least 95%, at least 96%, at least 97%, at least 98%, at least 99%, or 100% identity to SEQ ID NO: 16.

The fusion protein as described herein may have a V_{H1} comprising at least 90%, at least 91%, at least 92%, at least 93%, at least 94%, at least 95%, at least 96%, at least 97%, at least 98%, at least 99%, or 100% identity to SEQ ID NO: 17.

The fusion protein as described herein may have a V_{H1} comprising at least 90%, at least 91%, at least 92%, at least 93%, at least 94%, at least 95%, at least 96%, at least 97%, at least 98%, at least 99% or 100% identity to SEQ ID NO: 18.

The fusion protein as described herein may have a V_{H1} comprising at least 90%, at least 91%, at least 92%, at least 93%, at least 94%, at least 95%, at least 96%, at least 97%, at least 98%, at least 99% or 100% identity to SEQ ID NO: 19.

The terms “peptide”, “protein” and “polypeptide” are used interchangeably herein. The N-terminus of a protein (also known as the amino-terminus, NH₂-terminus, N-terminal end or amine-terminus) is the start of a protein or polypeptide terminated by an amino acid with a free amine group (—NH₂). By convention, peptide sequences are written N-terminus to C-terminus (from left to right). The C-terminus (also known as the carboxyl-terminus, carboxy-terminus, C-terminal tail, C-terminal end, or COOH-terminus) is the end of an amino acid chain (protein or polypeptide), terminated by a free carboxyl group (—COOH).

The term “fusion protein” as used herein is used to describe a protein that is created by joining at least two or more genes that originally encoded for at least two different proteins. Therefore, the fusion proteins described herein are synthetic and are not found in nature.

A fusion protein as described herein may comprise an amino acid sequence having at least 90%, at least 91%, at least 92%, at least 93%, at least 94%, at least 95%, at least 96%, at least 97%, at least 98%, at least 99% or 100% identity to SEQ ID NO: 1, 2, 3, 4, 5, 6, 7, 8, 9, 10, 11, 12, 13, 14, 15, 16, 17, 18, 19, 20, 21, 22 or 23. Fragments or portions of any sequence described herein are also encompassed. Percentage identity can be calculated as the percentage of identity to the entire length of the reference sequence (e.g. the sequence of SEQ ID NO:1 to 32), or portions or fragments thereof.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes can comprise at least 30%, preferably at least 40%, more preferably at least 50%, even more preferably at least 60%, and even more preferably at least 70%, at least 75%, at least 80%, at least 82%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970) J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

Alternatively, the percent identity between two amino acid or nucleotide sequences can be determined using the algorithm of Meyers et al. (1989) CABIOS 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-410). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used. See <http://www.ncbi.nlm.nih.gov>.

The polypeptides described herein can have amino acid sequences sufficiently or substantially identical to the amino acid sequences of SEQ ID NO:1 to 23. The terms “sufficiently identical” or “substantially identical” are used herein to refer to a first amino acid or nucleotide sequence that contains a sufficient or minimum number of identical or equivalent (e.g. with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least 90%, at least 91%, at least 92%, at least 93%, at least 94%, at least 95%, at least 96%, at least 97%, at least 98% or at least 99% identity are defined herein as sufficiently or substantially identical.

“Homology” or “identity” or “similarity” refer to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

The term “hybridising” means annealing to two at least partially complementary nucleotide sequences in a hybridization process. In order to allow hybridisation to occur complementary nucleic acid molecules are generally thermally or chemically denatured to melt a double strand into two single strands and/or to remove hairpins or other secondary structures from single-stranded nucleic acids. The stringency of hybridisation is influenced by conditions such as temperature, salt concentration and hybridisation buffer composition. Conventional hybridisation conditions are described in, for example, Sambrook (2001) Molecular Cloning: a laboratory manual, 3rd Edition Cold Spring Harbor Laboratory Press, CSH, New York, but the skilled craftsman will appreciate that numerous different hybridisation conditions can be designed in function of the known or the expected homology and/or length of the nucleic acid sequence. High stringency conditions for hybridisation include high temperature and/or low sodium/salt concentration (salts include sodium as for example in NaCl and Na-citrate) and/or the inclusion of formamide in the hybridisation buffer and/or lowering the concentration of compounds such as SDS (sodium dodecyl sulphate detergent) in the hybridisation buffer and/or exclusion of compounds such as dextran sulphate or polyethylene glycol (promoting molecular crowding) from the hybridisation buffer. By way of non-limiting example, representative salt and temperature conditions for stringent hybridization are: 1×SSC, 0.5% SDS at 65° C. The abbreviation SSC refers to a buffer used in nucleic acid hybridization solutions. One litre of a 20× (twenty times concentrate) stock SSC buffer solution (pH 7.0) contains 175.3 g sodium chloride and 88.2 g sodium citrate. A representative time period for achieving hybridisation is 12 hours.

A “non-essential” or “non-critical” amino acid residue is a residue that can be altered from the wild-type sequence of (e.g., the sequence of SEQ ID NO:1 to 23) without abolishing or, more preferably, without substantially altering a biological activity, whereas an “essential” amino acid residue results in such a change. For example, amino acid residues that are conserved among the polypeptides of the present invention are predicted to be particularly non-amenable to alteration, except that amino acid residues in transmembrane domains can generally be replaced by other residues having approximately equivalent hydrophobicity without significantly altering activity.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in protein is preferably replaced with another amino acid residue from the same side chain family. Alternatively, in another embodiment, mutations can be introduced randomly along all or part of coding sequences, such as by saturation mutagenesis, and the resultant mutants can be screened for biological activity to identify mutants that retain activity. Following mutagenesis, the encoded proteins can be expressed recombinantly and the biological activity of the protein can be determined.

As discussed above, the fusion protein described herein comprises at least two antibody V_{H} domains and at least two antibody V_{L} domains to provide two distinct binding sites comprising both V_{H} and V_{L} domains for an epitope employing techniques well known in the art (Biochim. Biophys. Acta, 192 (1969) 277-285; Proc. Natl. Acad. Sci. USA, Vol. 89, pp 10026-10030, November 1992). One or more CDRs may be taken from the described V_{H} domain and incorporated into a suitable framework comprising the primary structural conformation of amino acids as represented by one or more CDRs, optionally together with further amino acids which may enhance the binding affinity of one or more CDRs for the glycosylated receptor.

As described herein, the fusion protein comprises V_{H1} and V_{L1} that are connected by a first linker (L₁) as well as V_{H2} and V_{L2} that are connected by a second linker (L₂); in addition to the Fc. As used herein, the linker is a “domain linker”, which is used to combine any two domains as outlined herein together (e.g. V_{H1} and V_{L1} and/or V_{H2} and V_{L2}). Each of L₁ and L₂ may be a peptide linker. The linker peptide may predominantly include the following amino acid residues: Gly, Ser, Ala, or Thr. The linker peptide has a length that adequately links V_{H} and V_{L} in such a way that they assume the correct conformation relative to one another so that they retain their biological activity. The linker may be from about 1 to 50 amino acids in length. The linker may be from about 1 to 30 amino acids in length. The linker can be of 1 to 20 amino acids in length, optionally from about 5 to about 10 amino acids. Useful linkers include glycine-serine polymers, including for example (GS)n, (GSGGS)n, (GGGGS), and (GGGS)n, where n is an integer of at least one (and generally from 3 to 4), glycine-alanine polymers, alanine-serine polymers, and other flexible linkers. Alternatively, the linker may be a non-proteinaceous linker. A variety of non-proteinaceous polymers such as polyethylene glycol (PEG), polypropylene glycol, polyoxyalkylenes, or copolymers of polyethylene glycol and polypropylene glycol, may also be used as the non-proteinaceous linker. Other suitable linkers are well known in the art and may additionally or alternatively be used.

The linker L₁ and L₂ can comprise different linkers. Alternatively, L₁ and L₂ can comprise identical linkers. The linker L₁ can be at least 90%, at least 91%, at least 92%, at least 93%, at least 94%, at least 95%, at least 96%, at least 97%, at least 98%, at least 99% or 100% identical to GGGGSGGGGSGGGG (SEQ ID NO: 20). L₂ can be at least 90%, at least 91%, at least 92%, at least 93%, at least 94%, at least 95%, at least 96%, at least 97%, at least 98%, at least 99% or 100% identical to GGGGSGGGGSGGGG (SEQ ID NO: 20).

As described herein the fusion protein also comprises an Fc, which is also derived from antibodies. The terms “Fc” or “Fc region” or “Fc domain” are used interchangeably herein. By “Fc” or “Fc region” or “Fc domain” is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgG, the Fc domain comprises immunoglobulin domains Cy2 and Cy3 (Cy2 and Cy3) and the lower hinge region between Cy1 (Cy1) and Cy2 (Cy2). Although the boundaries of the Fc region may vary, the human IgG heavy chain Fc region is usually defined to include residues C226 or P230 to its carboxyl-terminus, wherein the numbering is according to the EU index as in Kabat. Amino acid modifications can be made to the Fc region, which are referred to as an Fc variant. Therefore, the fusion protein may have an Fc variant instead of an Fc. “Fc variant” or “variant Fc” as used herein is defined as a protein comprising an amino acid modification in an Fc domain.

The fusion protein could comprise an IgE Fc. Alternatively, the fusion protein may comprise an IgM Fc. The fusion protein may also comprise an IgA Fc. Finally, the fusion protein may comprise an IgG Fc. If the fusion protein does comprise an IgG Fc it may be an IgG1. Alternatively, it could be an IgG2. As s further alternative is could be an IgG3. As a final alternative, it could be an IgG4. Preferably, the Fc is an IgG Fc, most preferably and IgG1 Fc.

A typical IgG antibody is composed of two light and two heavy chains that are associated with each other to form three major domains connected through a flexible hinge region: the two identical antigen-binding (Fab) regions and the constant (Fc) region. The IgG-Fc region is a homodimer in which the two C_{H}3 domains are paired through non-covalent interactions. The two CH2 domains are not paired but each has a conserved N-glycosylation site at Asn-297. When the antibody recognizes and binds to a target cell, antibody-dependent cell-mediated cytotoxicity and other effector functions are triggered through the binding of the binding of the antibody's Fc region to ligands such as FcgR's on effector cells. The IgG-Fc N-glycan exists naturally as a bi-antennary complex having considerable heterogeneity. The different IgG-Fc glycosylation states have been shown to elicit significantly different effector functions. Studies have shown that the core structure of the N297-glycan, known as Man3GlcNAc2, particularly the initial three residues, is essential to conger significant stability and effector activity of antibody IgG-Fc (Jefferis et al., Biotechnol Prog 21:11-16). Moreover, recent structural studies have shown that the N-glycan might exert its effects mainly through stabilization of the Fc domain's conformation (Mimura et al., Mol Immunol 37:697-706; Krapp et al., J Mol Biol 325:979-989).

The Fc can also be defined according to its sequence. Therefore, the fusion protein as described herein may have an Fc comprising at least 90%, at least 91%, at least 92%, at least 93%, at least 94%, at least 95%, at least 96%, at least 97%, at least 98 or at least 99% identity to SEQ ID NO: 22. In another instance, the fusion protein as described herein may have an Fc comprising 100% identity to SEQ ID NO: 22.

The fusion protein as described herein may further comprise a third linker (L₃) and a fourth linker (L₄) which connect the Fc described herein to any one of the V_{H1}, V_{H2}, V_{L1}, and V_{L2} domains of the fusion protein. L₃ and L₄ may also be used interchangeably with L₁ and L₂ as described above.

The L₃ and L₃ linkers may comprise non-identical sequences. Alternatively, the L₃ and L₄ linkers may comprise identical sequences. More specifically, the L₃ and L₄ may comprise at least 90%, at least 91%, at least 92%, at least 93%, at least 94%, at least 95%, at least 96%, at least 97%, at least 98% or at least 99% identity to SEQ ID NO: 21. The L₃ and L₄ may comprise 100% identity to SEQ ID NO: 21. The L₃ and L₄ may consist of SEQ ID NO: 21

The fusion protein as described herein, may have a sequence comprising at least 90%, at least 91%, at least 92%, at least 93%, at least 94%, at least 95%, at least 96%, at least 97%, at least 98% or at least 99% identity to SEQ ID NO: 23. The fusion protein as described herein, may alternatively have a sequence comprising 100% identity to SEQ ID NO: 23. Most specifically, the fusion protein as described herein may consist of SEQ ID NO: 23.

The fusion protein, as described herein, may further comprise a signal peptide. As used herein “signal peptide” refers to a short peptide typically located at the amino-terminal of the fusion protein. The signal peptide functions to target the fusion protein towards the secretory pathway of a cell once it is delivered to a cell. Several different signal peptide sequences are well known in the art. The type of signal peptide used can depend to some extent on the type (prokaryote vs eukaryote) of host cell that is used for fusion protein production.

For example, in eukaryotes, a signal peptide that contains 5-30 amino acids present at the N-terminus of nascent proteins is recognized by the signal recognition particle (SRP) in the cytosol while the protein is still being synthesized on the ribosome. The SRP then delivers the SRP-ribosome-nascent chain (SRP-RNC) complex to the SRP-receptor (SR) in the endoplasmic reticulum (ER) membrane. GTP-dependent mechanisms then deliver the RNC complex to a membrane-bound translocon which allows translocation of the growing polypeptide chain into the lumen of the ER. After crossing the ER membrane, the signal peptide is cleaved off by a signal peptide peptidase (SPP). Several signal peptides suitable for protein expression in eukaryotic cells are known. An example of a signal peptide that has been used in cells include MEWSWVFLFFLSVTTGVHS [SEQ ID NO: 32].

As described above, the terms “peptide”, “protein” and “polypeptide” are used interchangeably herein. The N-terminus of a protein (also known as the amino-terminus, NH₂-terminus, N-terminal end or amine-terminus) is the start of a protein or polypeptide terminated by an amino acid with a free amine group (—NH₂). By convention, peptide sequences are written N-terminus to C-terminus (from left to right). The C-terminus (also known as the carboxyl-terminus, carboxy-terminus, C-terminal tail, C-terminal end, or COOH-terminus) is the end of an amino acid chain (protein or polypeptide), terminated by a free carboxyl group (—COOH).

As used herein, the terms “amino-terminal” and “carboxy-terminal” are used to describe the relative position of e.g. a signal peptide or linker or domain within a polypeptide. Accordingly, a signal peptide or linker or domain that is “amino-terminal” is positioned closer (in relative terms) to the amino-terminus than to the carboxy-terminus of the polypeptide. Conversely, a signal peptide or linker or domain that is “carboxy-terminal” is positioned (in relative terms) closer to the carboxy-terminus than to the amino-terminus of the polypeptide. As used herein, the term “positioned” refers to the location of the e.g. signal peptide, linker or domain within the linear amino acid sequence of the polypeptide.

The terms “amino-terminal” and “carboxy-terminal” can be used to describe the relative position of two or more linkers or domains within a polypeptide. In this context, a linker or domain that is “amino-terminal” is positioned closer (in relative terms) to the amino-terminus of the polypeptide than a linker or domain that is “carboxy-terminal”. Conversely, a linker or domain that is “carboxy-terminal” is positioned closer (in relative terms) to the carboxy-terminus of the polypeptide than a linker or domain that is “amino-terminal”.

A linker, signal peptide or domain that is “amino-terminal” may be, but does not have to be, at the amino-terminus of the polypeptide (i.e. it may be, but does not have to be, at the start of the polypeptide terminated by an amino acid with a free amine group). In other words, the first amino acid of an amino-terminal linker, signal peptide or domain does not need to be (but may be) the first amino acid of the polypeptide. This means that there may be other amino acids, polypeptide linkers, signal peptides or domains (e.g. signal, peptides, tags such as HA tags) etc between the amino-terminus of the polypeptide and the start of the “amino-terminal” linker, signal peptide or domain (provided that the linker, signal peptide or domain is positioned closer to the amino-terminus than to the carboxy-terminus of the polypeptide; or when used to describe the relative positions of two or more linker, signal peptide or domains, provided that the linker, signal peptide or domains are positioned closer to the amino-terminus than a part that is “carboxy-terminal”).

Likewise, a linker, signal peptide or domain that is “carboxy-terminal” may be, but does not have to be, at the carboxy-terminus of the polypeptide (i.e. it may be, but does not have to be, at the end of the polypeptide terminated by any amino acid with a free carboxyl group). In other words, the last amino acid of a carboxy-terminal domain does not need to be (but may be) the last amino acid of the polypeptide. This means that there may be other amino acids, polypeptide parts etc (e.g. tags) between the carboxy-terminus of the polypeptide and the end of the “carboxy-terminal” linker, signal peptide or domain (provided that the linker, signal peptide or domain is positioned closer to the carboxy-terminus than to the amino-terminus of the polypeptide; or when used to describe the relative positions of two or more linkers or domains, provided that the linkers or domains are positioned closer to the carboxy-terminus than a linker or domains that is “amino-terminal”).

Polypeptides comprising an amino-terminal polypeptide part (A) and a carboxy-terminal polypeptide part (B) are conventionally written as A-B i.e. N-terminal to C-terminal (left to right).

By way of example, a polypeptide comprising an amino-terminal signal peptide part (S) and a carboxy-terminal V_{H1} part will be conventionally written as (S)-(V_{H1}) or SV_{H1}. The signal peptide described herein may be upstream of the amino-terminus of the sequence amino-terminus of V_{H1}. The signal peptide described herein may be upstream of the amino-terminus of the sequence amino terminus of V_{H2}.

During maturation of the fusion protein, the signal peptide is cleaved/processed. Therefore, the fusion protein may be free from an additional amino-terminal sequence, such as the signal peptide.

For example, when L₁ is located downstream of the carboxy terminus of V_{H1} and upstream of the amino terminus of V_{L1} and/or wherein L₂ is located downstream of the carboxy terminus of V_{H2} and upstream of the amino terminus of V_{L2}.

Therefore, the fusion protein as described herein may have an arrangement of (V_{H1})-(L₁)-(V_{L1})-(Fc)-(V_{H2})-(L₂)-(V_{L2}). Alternatively, the fusion protein as described herein may have an arrangement of (S)-(V_{H1})-(L₁)-(V_{L1})-(Fc)-(V_{H2})-(L₂)-(V_{L2}). The fusion protein as described herein may have an arrangement of (V_{H1})-(L₁)-(V_{L1})-(L₃)-(Fc)-(L₄)-(V_{H2})-(L₂)-(V_{L2}). The fusion protein as described herein could have an arrangement of (S)-(V_{H1})-(L₁)-(V_{L1})-(L₃)-(Fc)-(L₄)-(V_{H2})-(L₂)-(V_{L2}). The fusion protein as described herein may have an arrangement of (V_{H2})-(L₂)-(V_{L2})—(Fc)-(V_{H1})-(L₁)-(V_{L1}). The fusion protein as described herein can also have an arrangement of (S)-(V_{H2})-(L₂)-(V_{L2})-(Fc)-(V_{H1})-(L₁)-(V_{L1}). The fusion protein as described herein may have an arrangement of (V_{H2})-(L₁)-(V_{L2})-(L₃)-(Fc)-(L₄)-(V_{H1})-(L₂)-(V_{L1}). Finally, the fusion protein as described herein may have an arrangement of (S)-(V_{H1})-(L₂)-(V_{L1})-(L₃)-(Fc)-(L₄)-(V_{H2})-(L₁)-(V_{L2}).

In one aspect, the fusion protein is a fusion protein comprising:
(a) a first binding domain;
(b) a second binding domain; and
(c) a human Fc domain (Fc);
   wherein each of the first and second binding domains selectively bind to Neu5Acα2-3Galβ1-3GalNAcα-R.

As used herein the term “specific” and “specifically” are used interchangeably to indicate that other biomolecules do not significantly bind to the binding domains that are binding to the biomolecule of interest (i.e. the epitope Neu5Acα2-3Galβ1-3GalNAcα-R). In some embodiments, the level of binding to a biomolecule other than the epitope Neu5Acα2-3Galβ1-3GalNAcα-R results in a negligible (e.g., not determinable) binding affinity by means of ELISA or an affinity determination.

By “negligible binding” a binding is meant, which is at least about 85%, particularly at least about 90%, more particularly at least about 95%, even more particularly at least about 98%, but especially at least about 99% and up to 100% less than the binding to a peptide comprising the epitope Neu5Acα2-3Galβ1-3GalNAcα-R.

The binding affinity of an antibody to a peptide or epitope may be determined with a standard binding assay, such as surface plasmon resonance technique (BIAcore®, GE-Healthcare Uppsala, Sweden). The term “surface plasmon resonance,” as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51: 19-26; Jonsson, U., et al. (1991) Biotechniques 11 :620-627; Johnsson, B., et al. (1995) J. Mol. Recognit. 8: 125-131; and Johnnson, B., et al. (1991) Anal. Biochem. 198:268-277.

As used herein the term “epitope” refers to a site on a target molecule (e.g., Neu5Acα2-3Galβ1-3GalNAcα-R) to which the binding domains of the fusion protein bind. Epitopes can be formed both from contiguous or adjacent noncontiguous residues (e.g., amino acid residues) of the target molecule. Preferably, the epitope of the present invention is Neu5Acα2-3Galβ1-3GalNAcα-R.

Also described herein is a nucleic acid encoding any of the fusion proteins described herein.

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesised) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear. The terms “polynucleotide” and “oligonucleotide” are used interchangeably and refer to a polymeric form of nucleotides of any length, either deoxyribonucleotides or ribonucleotides or analogs thereof.

The nucleic acid sequence as described herein may comprise at least 90% identity to SEQ ID NO: 24. The nucleic acid sequence as described herein may comprise at least 91% identity to SEQ ID NO: 24. The nucleic acid sequence as described herein may comprise at least 92% identity to SEQ ID NO: 24. The nucleic acid sequence as described herein may comprise at least 93% identity to SEQ ID NO: 24. The nucleic acid sequence as described herein may comprise at least 94% identity to SEQ ID NO: 24. The nucleic acid sequence as described herein may comprise at least 95% identity to SEQ ID NO: 24. The nucleic acid sequence as described herein may comprise at least 96% identity to SEQ ID NO: 24. The nucleic acid sequence as described herein may comprise at least 97% identity to SEQ ID NO: 24. The nucleic acid sequence as described herein may comprise at least 98% identity to SEQ ID NO: 24. The nucleic acid sequence as described herein may comprise at least 99% identity to SEQ ID NO: 24. The nucleic acid sequence as described herein may comprise 100% identity to SEQ ID NO: 24.

The signal peptide described herein may also be encoded by a nucleotide sequence. Therefore, the nucleic acid encoding the fusion protein may also further comprise a nucleotide sequence encoding any signal peptide described herein.

The nucleic acid encoding the fusion protein as described herein could be in the form of an expression cassette. The expression cassette will comprise a promoter operably linked to the nucleic acid described herein.

The term “operably linked”, “operably connected” or equivalent expressions as used herein refer to the arrangement of various nucleic acid elements relative to each such that the elements are functionally connected and are able to interact with each other in the manner intended. Such elements may include, without limitation, a promoter, an enhancer and/or a regulatory element, a polyadenylation sequence, one or more introns and/or exons, and a coding sequence of a gene of interest to be expressed. The nucleic acid sequence elements, when properly oriented or operably linked, act together to modulate the activity of one another, and ultimately may affect the level of expression of an expression product. By modulate is meant increasing, decreasing, or maintaining the level of activity of a particular element. The position of each element relative to other elements may be expressed in terms of the 5′ terminus and the 3′ terminus of each element, and the distance between any particular elements may be referenced by the number of intervening nucleotides, or base pairs, between the elements. As understood by the skilled person, operably linked implies functional activity, and is not necessarily related to a natural positional link. Indeed, when used in nucleic acid expression cassettes, cis-regulatory elements will typically be located immediately upstream of the promoter (although this is generally the case, it should definitely not be interpreted as a limitation or exclusion of positions within the nucleic acid expression cassette), but this needs not be the case in vivo, e.g., a regulatory element sequence naturally occurring downstream of a gene whose transcription it affects is able to function in the same way when located upstream of the promoter. Hence, according to a specific embodiment, the regulatory or enhancing effect of the regulatory element is position-independent.

As used herein, the phrase “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of Transcription Factors (TFs). TFs bind to the promoter sequences and result in the recruitment of RNA polymerase, an enzyme that synthesizes RNA from the coding region of the gene. A great many promoters are known in the art.

The expression cassette may further comprise a nucleic acid encoding a post-transcriptional regulatory element. An example of a post-transcriptional regulatory element is a polyA element. Therefore, the expression cassette may further comprise a nucleic acid sequence encoding a polyA element.

The inventors have identified eight promoters of especial interest in a therapeutic context. These eight promoters are expected to work especially well in cancer cells and not in normal cells.

MUC1, LP, CEACAM5, COX2 and SURVIVIN are five promoters that are located upstream of genes that are known to be upregulated in cancer cells. Consequently, these promoters are expected to induce expression of the fusion protein when the expression cassette is introduced into a cancer cell. Therefore, the promoter in the expression cassette may be SEQ ID NO: 25. Alternatively, the promoter may be SEQ ID NO: 26. Alternatively, the promoter may be SEQ ID NO: 27. The promoter could also be SEQ ID NO: 28. The promoter could also be SEQ ID NO: 29.

Alternatively, the promoter could be SEQ ID NO: 3. This is the KAZALD1 promoter. The KAZALD1 promoter is selected because it is widely known to induce a high-level of expression in a wide variety of mammalian cells. The KAZALD1 promoter can be operably linked to a nucleic acid encoding a fusion protein as described herein. In one instance a nucleic acid comprising the KAZALD1 promoter operably linked to a nucleic acid encoding a fusion protein as described herein can be used for the treatment of brain cancer (also referred herein as glioblastoma herein) or cervical cancer. The aforementioned nucleic acid may be delivered via an adenoviral vector as described elsewhere herein.

PTP4A1 and SMYD5 are preferred as promoters because the genes that are downstream of these promoters were upregulated when the fusion protein is used to treat cancer cells. Therefore, in a therapeutic setting, if these genes are up-regulated their promoters will drive higher expression of the fusion protein. Thus, the promoter could also be SEQ ID NO: 31. Finally, the promoter could be SEQ ID NO: 32.

The expression cassette may comprise the nucleic acid described herein operably linked to the PTP4A1 promoter. The expression cassette may comprise the nucleic acid described herein operably linked to the SMYD5 promoter. The expression cassette may comprise the nucleic acid described herein operably linked to the MUC1 promoter. The expression cassette may comprise the nucleic acid described herein operably linked to the LP promoter. The expression cassette may comprise the nucleic acid described herein operably linked to the CEACAM5 promoter. The expression cassette may comprise the nucleic acid described herein operably linked to the COX2 promoter. The expression cassette may comprise the nucleic acid described herein operably linked to the SURVIVIN promoter. The expression cassette may finally comprise the nucleic acid described herein operably linked to the KAZALD1 promoter.

The expression cassette described herein may be incorporated into a vector.

The term “vector” is well known in the art, and as used herein refers to a nucleic acid molecule, e.g. double-stranded DNA, which may have inserted into it a nucleic acid sequence according to the present invention. A vector is used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a host cell, the vector can replicate independently of, or coincidental with, the host chromosomal DNA; several copies of the vector and its inserted nucleic acid molecule may be generated. Vectors of the present invention can be episomal vectors (i.e., that do not integrate into the genome of a host cell), or can be vectors that integrate into the host cell genome. This definition includes both non-viral and viral vectors. Non-viral vectors include but are not limited to plasmid vectors (e.g. pMA-RQ, pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Larger vectors such as artificial chromosomes (bacteria (BAC), yeast (YAC), or human (HAC)) may be used to accommodate larger inserts.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors (AAV), alphavirus vectors and the like. Typically, but not necessarily, viral vectors are replication-deficient as they have lost the ability to propagate in a given cell since viral genes essential for replication have been eliminated from the viral vector (e.g. replication deficient).

However, some viral vectors can also be adapted to replicate specifically in a given cell, such as e.g. a cancer cell, and are typically used to trigger the (cancer) cell-specific (onco)lysis. These viral vectors are referred to herein as “oncolytic viruses” or CRAds.

For example, normal dividing cells transiently lack the growth control mechanism, retinoblastoma (RB) tumor suppressor, that is lacking in and associated with unrestricted growth in certain neoplastic cells. The loss of retinoblastoma tumor suppressor gene (RB) gene function has been associated with the etiology of various types of tumors. The product of this tumor suppressor gene, a 105 kilodalton polypeptide called pRB or p 105, is a cell-cycle regulatory protein. The pRB polypeptide inhibits cell proliferation by arresting cells at the G-phase of the cell cycle. The pRB protein is a major target of several DNA virus oncoproteins, including adenovirus E1A, which binds and inactivates pRB, and this inactivation is important in facilitating viral replication. The pRB protein interacts with the E2F transcription factor, which is involved in the expression of the adenovirus E2 gene and several cellular genes, and inhibits the activity of this transcription factor (Bagchi et al. (1991) Cell 65: 1063: Bandara et al. (1991) Nature 351: 494; Chellappan et al. (1999) Proc. Natl. Acad. Sci. (U.S.A) 89: 4549).

Consequently, an adenovirus or AAV that is “replication defective” is used to refer to an adenoviral or AAV variant which lacks the capacity to complex with RB but substantially retains other essential replicative functions so as to exhibit a replication-competent phenotype in cells which are deficient in RB function (e. g., cells which are homozygous or heterozygous for substantially deleted RB alleles, cells which comprise RB alleles encoding mutant RB proteins which are essentially non-functional cells which comprise mutations that result in a lack of function of an RB protein) but will not substantially exhibit a replicative phenotype in non-replicating, non-cancerous cells. Such replication deficient adenovirus or AAV species may be referred to as Δ24 E1A replication deficient adenoviruses or AAV.

A functional E1 deletion or functional E3 deletion, among others may be selected. Additionally, a functional E4 deletion may also be selected. The term “functionally deleted” or “functional deletion” means that a sufficient amount of the gene region is removed or otherwise damaged, e.g., by mutation or modification, so that the gene region is no longer capable of producing functional products of gene expression.

Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

The viral vector described herein may comprise a nucleic acid described herein or an expression cassette described herein. Preferably, the viral vector is an adenoviral vector. The adenoviral vector may also be a hybrid vector, which is an artificial vector comprising components from different adenovirus sub-types. Therefore, the adenovirus may be a hybrid Ad5/35 viral vector. Alternatively, the adenovirus may be a hybrid Ad5/3 viral vector. The viral vector may also be an oncolytic vector. The adenoviral vector as described herein may have an E1 deletion. The adenoviral vector as described herein may have an E3 deletion. The adenoviral vector as described herein may have an E1 and E3 deletion.

The adenoviral vector described herein may be formulated as a pharmaceutical composition. The adenoviral vector composition could comprise a single type of andenoviral vector. Alternatively, the pharmaceutical composition may comprise a cocktail of adenoviral vectors which all comprise nucleic acids sequences encoding the fusion protein described herein but are operably linked to different promoters (i.e. different expression cassettes). Therefore, the cocktail of adenoviruses may comprise a plurality of expression cassettes selected from the list comprising of: the nucleic acid described herein operably linked to the PTP4A1 promoter; the nucleic acid described herein operably linked to the SMYD5 promoter; the nucleic acid described herein operably linked to the MUC1 promoter; the nucleic acid described herein operably linked to the LP promoter; comprise the nucleic acid described herein operably linked to the CEACAM5 promoter; the nucleic acid described herein operably linked to the COX2 promoter; the nucleic acid described herein operably linked to the SURVIVIN promoter or the nucleic acid described herein operably linked to the KAZALD1 promoter.

Alternatively, the cocktail of adenoviruses may comprise two expression cassettes selected from the list comprising: the nucleic acid described herein operably linked to the PTP4A1 promoter; the nucleic acid described herein operably linked to the SMYD5 promoter; the nucleic acid described herein operably linked to the MUC1 promoter; the nucleic acid described herein operably linked to the LP promoter; comprise the nucleic acid described herein operably linked to the CEACAM5 promoter; the nucleic acid described herein operably linked to the COX2 promoter; the nucleic acid described herein operably linked to the SURVIVIN promoter or the nucleic acid described herein operably linked to the KAZALD1 promoter.

Alternatively, the cocktail of adenoviruses may comprise three expression cassettes selected from the list comprising: the nucleic acid described herein operably linked to the PTP4A1 promoter; the nucleic acid described herein operably linked to the SMYD5 promoter; the nucleic acid described herein operably linked to the MUC1 promoter; the nucleic acid described herein operably linked to the LP promoter; comprise the nucleic acid described herein operably linked to the CEACAM5 promoter; the nucleic acid described herein operably linked to the COX2 promoter; the nucleic acid described herein operably linked to the SURVIVIN promoter or the nucleic acid described herein operably linked to the KAZALD1 promoter.

Alternatively, the cocktail of adenoviruses may comprise four expression cassettes selected from the list comprising: the nucleic acid described herein operably linked to the PTP4A1 promoter; the nucleic acid described herein operably linked to the SMYD5 promoter; the nucleic acid described herein operably linked to the MUC1 promoter; the nucleic acid described herein operably linked to the LP promoter; comprise the nucleic acid described herein operably linked to the CEACAM5 promoter; the nucleic acid described herein operably linked to the COX2 promoter; the nucleic acid described herein operably linked to the SURVIVIN promoter or the nucleic acid described herein operably linked to the KAZALD1 promoter.

Alternatively, the cocktail of adenoviruses may comprise five expression cassettes selected from the list comprising: the nucleic acid described herein operably linked to the PTP4A1 promoter; the nucleic acid described herein operably linked to the SMYD5 promoter; the nucleic acid described herein operably linked to the MUC1 promoter; the nucleic acid described herein operably linked to the LP promoter; comprise the nucleic acid described herein operably linked to the CEACAM5 promoter; the nucleic acid described herein operably linked to the COX2 promoter; the nucleic acid described herein operably linked to the SURVIVIN promoter or the nucleic acid described herein operably linked to the KAZALD1 promoter.

Alternatively, the cocktail of adenoviruses may comprise six expression cassettes selected from the list comprising: the nucleic acid described herein operably linked to the PTP4A1 promoter; the nucleic acid described herein operably linked to the SMYD5 promoter; the nucleic acid described herein operably linked to the MUC1 promoter; the nucleic acid described herein operably linked to the LP promoter; comprise the nucleic acid described herein operably linked to the CEACAM5 promoter; the nucleic acid described herein operably linked to the COX2 promoter; the nucleic acid described herein operably linked to the SURVIVIN promoter or the nucleic acid described herein operably linked to the KAZALD1 promoter.

Alternatively, the cocktail of adenoviruses may comprise seven expression cassettes selected from the list comprising: the nucleic acid described herein operably linked to the PTP4A1 promoter; the nucleic acid described herein operably linked to the SMYD5 promoter; the nucleic acid described herein operably linked to the MUC1 promoter; the nucleic acid described herein operably linked to the LP promoter; comprise the nucleic acid described herein operably linked to the CEACAM5 promoter; the nucleic acid described herein operably linked to the COX2 promoter; the nucleic acid described herein operably linked to the SURVIVIN promoter or the nucleic acid described herein operably linked to the KAZALD1 promoter.

Alternatively, the cocktail of adenoviruses may comprise eight expression cassettes comprising: the nucleic acid described herein operably linked to the PTP4A1 promoter; the nucleic acid described herein operably linked to the SMYD5 promoter; the nucleic acid described herein operably linked to the MUC1 promoter; the nucleic acid described herein operably linked to the LP promoter; comprise the nucleic acid described herein operably linked to the CEACAM5 promoter; the nucleic acid described herein operably linked to the COX2 promoter; the nucleic acid described herein operably linked to the SURVIVIN promoter or the nucleic acid described herein operably linked to the KAZALD1 promoter.

For the avoidance of doubt, the adenoviral vector need not be the same adenoviral vector and therefore it is contemplated that a pharmaceutical composition may comprise a plurality of expression cassettes but also a plurality of adenoviral vectors.

In another aspect, the fusion protein described herein, nucleic acid described herein, expression cassette described herein and adenoviral vectors described herein are used to treat a disease or in methods of treatment.

Accordingly, there is provided a method for treating a disease, wherein the method comprises: administering a therapeutically effective amount of the fusion protein described herein, nucleic acid described herein, expression cassette described herein or the adenoviral vectors described herein.

As used herein, the “administration” or “administering” of an agent (e.g., a fusion protein, expression cassette, viral particle, vector, polynucleotide, cell, population of cells, composition, or pharmaceutical composition) to a subject includes any route of introducing or delivering to a subject the agent to perform its intended function. Administration can be carried out by any suitable route, including intratumorally, orally, intranasally, intraocularly, ophthalmically, parenterally (intravenously, intramuscularly, intraperitoneally, or subcutaneously), or topically. Administration includes self-administration and the administration by another.

As used herein, the phrases “therapeutically effective amount” means a dose or plasma concentration in a subject that provides the specific pharmacological effect for which the disclosed vectors are administered, e.g. to treat a disease of interest in a target subject. The therapeutically effective amount may vary based on the route of administration and dosage form, the age and weight of the subject, and/or the disease or condition being treated.

As used herein, the terms “treatment” or “treating” refer to reducing, ameliorating or eliminating one or more signs, symptoms, or effects of a disease or condition.

As used herein, a “subject” refers to a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate, e.g., a human. A subject can be male or female. A subject can be a fully developed subject (e.g., an adult) or a subject undergoing the developmental process (e.g., a child or infant). The subject can be a mammal. The mammal can be a human, non-human primate, mouse, rat, dog, cat, horse, or cow, but are not limited to these examples. Preferably the subject is human.

When used as a treatment, the fusion protein described herein, nucleic acid described herein, expression cassette described herein or the adenoviral vectors described herein can be formulated as a pharmaceutically acceptable composition.

A pharmaceutical composition is also provided herein, wherein the composition comprises an adenovirus, genotype, isolated nucleic acid sequence, vector or protein and a pharmaceutically acceptable excipient, adjuvant, diluent and/or carrier. Compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected binding protein without causing any undesirable biological effects or interacting in a deleterious manner with any of the other components of the pharmaceutical composition in which it is contained.

Excipients are natural or synthetic substances formulated alongside an active ingredient (e.g. an adenovirus, genotype, isolated nucleic acid sequence, vector or protein), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art.

The pharmaceutical compositions described herein may be used for targeting to any cells with a receptor for adenoviruses.

In preferred aspects, the disease to be treated is cancer.

In even more preferred aspects the cancer is selected from breast, triple-negative breast, melanoma, lung, small cell lung, B-cell leukemia, prostate, bladder, colon, glioblastoma, liver, cervical, ovarian and head and neck cancer.

Another object of the present invention is producing a binding molecule and more specifically antibodies or fragments of antibodies that can distinguish between glycosylated proteins and glycosylated sphingolipids.

### EXAMPLES

### Example 1—Generation of Monoclonal Antibodies

Methods for production of monoclonal antibodies are well known in the art, they mainly include the formation of hybridoma cells lines formed from the fusion of murine spleen cells from animals that have been immunized with the desired antigen and myeloma cells. Antibodies produced form the hybridoma cells are purified and can be used as diagnostic tools or for treatment.

Production of monoclonal antibodies using hybridoma cell lines is a technique well known in the art. Is has been specifically described elsewhere (Goding J. W. Monoclonal antibodies: principles and practice: production and application of monoclonal antibodies in cell biology, biochemistry and immunology. 2^{nd} edition. 1986 Academic Press, Harcour Brace Jovnovich Publishers). In accordance with conventional techniques, a key step in this process has been to screen and to select for hybridomas for antibody production which that produce supernatants having affinity for the peptide immunogen against which they have been raised.

A sequence within the carboxyl-terminal region of the human P2X7 receptor was selected for use as the antigen for monoclonal antibody generation.

The carboxylic-terminal cytoplasmic domains of P2X7 receptor comprise amino acids 352 to 595 and are longer than in other members of the P2X subtype. This domain is crucial for P2X7 pore formation, transduction and signalling. Allelic mutations, leading to loss of function, have been identified in the human and mouse receptor. It has been suggested that pore formation requires over 95% of the C-terminal tail of the receptor.

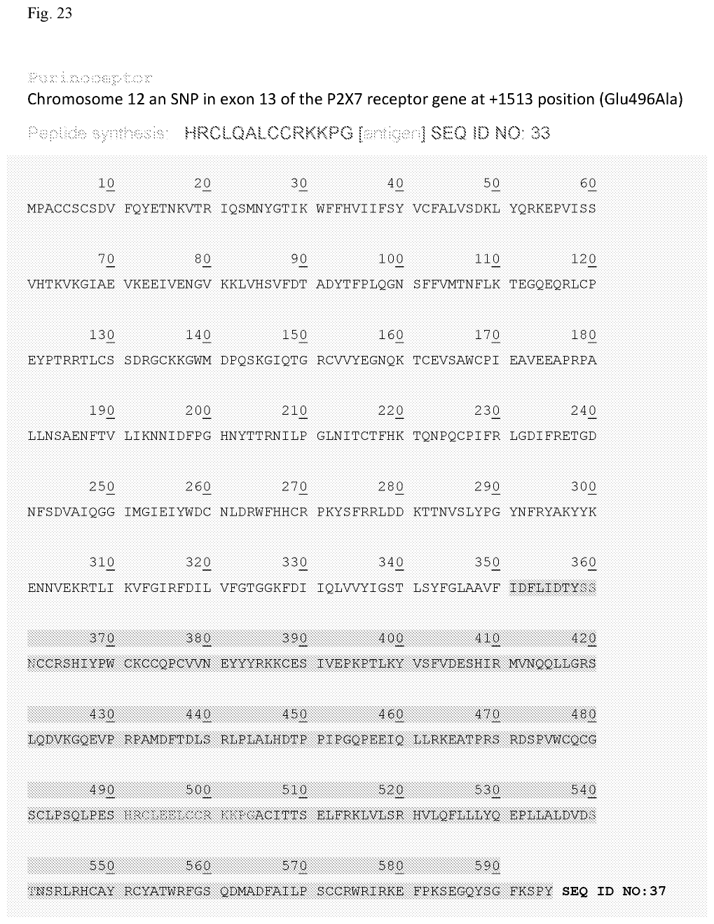

The full-length polypeptides sequence of the human P2X7 receptor is shown in FIG. 23.

The wild type antigen sequence HRCLEELCCRKKPG is common in both human and mouse species and was selected as the antigen. The antigen sequence is located at position 491-504 of the polypeptide sequence (underlined on the sequence above) However, for antibody generation the wild type sequence was modified such that antigen of the invention included following substitutions E495Q and E496A, to provide modified antigen sequence HRCLQALCCRKKPG.

The antigen and epitope are similar but not the exact same amino acid sequence. The epitope is discontinuous and part of a post translational modified P2X7 receptor common marker.

For the generation of monoclonal antibodies, hybridoma cell lines expressing the desired antibody were produced. Techniques for the production of monoclonal antibodies are well known in the art. Animals were presented with the carboxyl-terminal antigen sequence HRCLQALCCRKKPG conjugated to KHL. Murine hybridoma cells were produced and screened according to standard protocols. Monoclonal antibodies harvested after clonal selection of the hybridomas expressed from the variable regions of the monoclonal antibodies raised against the antigen, were tested for their capacity to bind this antigen. The monoclonal antibodies were cross-reacted in-vitro, against a synthetic peptide, HRCLQQLCCRKKPG, the wild type SNP at position 496 which is Q. Binding molecules which did not react with the sequence HRCLQQLCCRKKPG were selected.

Cross reacting epitope of KLH-Gal(ß1-3)GalNAc-Nana—determinants were thus obtained which represent the sugar biomarkers in cancer.

The fusion protein created from the MABs have low selectivity toward the P2X7 pore, but high selectivity for glycocalyx specific for cancer cells.

The antibodies each selectively bind the common epitope:

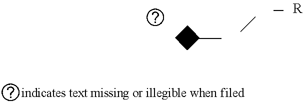

wherein R is a peptide sequence having a site capable of glycosylation with an O-glycan. In one example the peptide sequence is glycosylated with an O-glycan. In a further example, the peptide can contain a serine and/or threonine with an O-glycan glycosylation.

### Example 2—Determination of Variable Region Sequences

The next step is the determination of the variable region sequences from monoclonal antibodies identified in Example 1. Three hybridomas, from a total of nineteen, each producing an antibody capable of forming an immune complex with sialosylated glycosphingolipids and sialated glycoproteins as well as their independent constituent monosaccharide sugars namely neu5ac galnac and gal that constitute the glycocalyx.

The three hybridomas secrete monoclonal antibodies that bind to the 160 proteins at 80 KDa and 100 KDa in 4 breast cancer cell lines but not in MCF10A normal breast cell line.

Furthermore, the variable region sequences from the three monoclonal antibodies secreted by hybridomas were sequenced using techniques known in the art. The employed protocol involved:
1. Extract and purify total RNA from hybridoma cells or clonal B cell.
2. Synthesize cDNA using optimized primer set.
3. Amplify the DNA fragments corresponding to the VL and VH using nested PCR.
4. Sequence the amplified DNA fragments.

In particular, the total RNA was extracted from the hybridoma cells using a QIAGEN RNeasy Mini Kit (Cat. No. 74104), according to manufacturer's instructions. RT-PCR was performed using a QIAGEN RT-PCR Kit (Cat. No. 210210), according to manufacturer's instructions. Finally, the RACE method (Rapid Amplification of cDNA Ends) was used according to standard molecular biology protocols.

The three clones are clone 1: 7F11.B9 Isotype Ig1 k. Clone 2: 2G7.A8 Isotype Ig1 k. Clone 3: 6H5.C10 Isotype Ig1 k.

### Example 3—Induction of Apoptosis

The three hybridomas of Example 2, each producing a monoclonal antibody capable of apoptosis in cancer cell lines, were used in Caspase 3 signal transduction studies. In particular, the produced antibodies from the three hybridomas were incubated with living MCF7 cells and the percentage of apoptosis induced was measured.

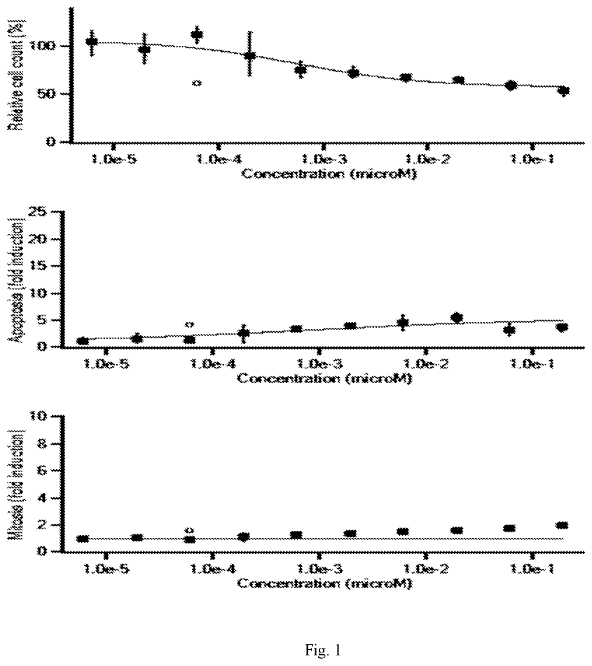

More specifically, living MCF7 cells were seeded in 384 well plates at one cell density in standardized media and 24 hrs after plating the cells were incubated with the antibody produced by the 2G7.A8 hybridoma clone of example 2 at various concentrations and over the following time course: 90 min, 3 hr, 6 hr, 24 hr, 72 hr at 37° C. For the purpose of the present invention, the antibodies produced by the 2G7.A8 clone will be the 2G7.A8 mAb. The cells were then fixed according to standard protocols and stained with DAPI to visualize the nucleus and anti-cleaved caspase3/7 to visualize apoptotic cells. Fluorescence was measured by an Image Xpress Micro high content fluorescence imager to acquire all of the data. Cell count was measured with nuclear dye DAPI, excitation max 350 nm. Binding of antibody was detected with an orange fluorescent antibody, excitation max 594 nm. Apoptosis was detected with a green fluorescent antibody, excited at 490 nm. (Table 1 and FIG. 1). Significant statistical values indicative of apoptosis are highlighted in table 1 below.

TABLE 1
Apoptosis measurements in MCF7 cells incubated
with the antibodies of example 2.

| Concentration | Relative cell count (%) | | Apoptosis (fold induction) | |
|---|---|---|---|---|
| (microM) | Mean | StdDev | Mean | StdDev |
| 6.16E−06 | 104.9 | 12.0 | 1.1 | 0.6 |
| 1.95E−05 | 96.1 | 14.5 | 1.6 | 0.8 |
| 6.15E−05 | 112.0 | 8.8 | 1.3 | 0.3 |
| 1.94E−04 | 90.4 | 21.6 | 2.7 | 1.5 |
| 6.14E−04 | 75.3 | 7.7 | 3.4 | 0.4 |
| 1.94E−03 | 72.2 | 5.3 | 4.0 | 0.5 |
| 6.13E−03 | 67.4 | 3.3 | 4.6 | 1.4 |
| 1.94E−02 | 65.1 | 3.1 | 5.4 | 0.8 |
| 6.12E−02 | 59.4 | 4.4 | 3.2 | 1.1 |
| 1.94E−01 | 54.1 | 4.4 | 3.7 | 0.6 |

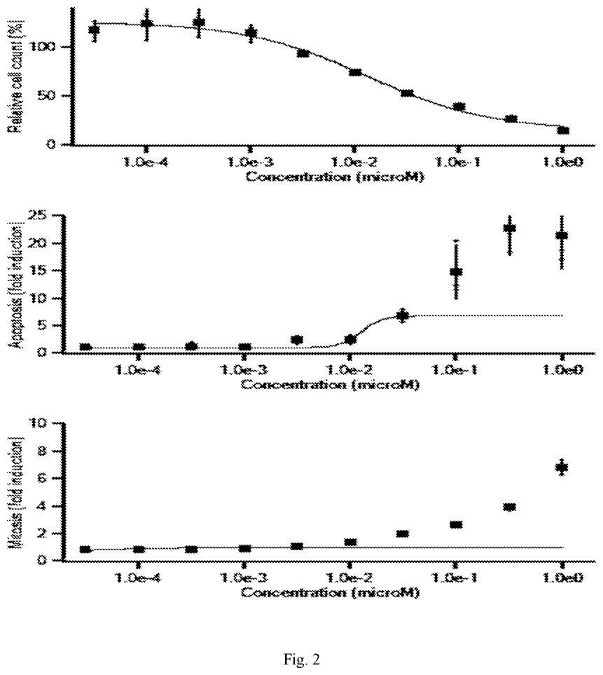

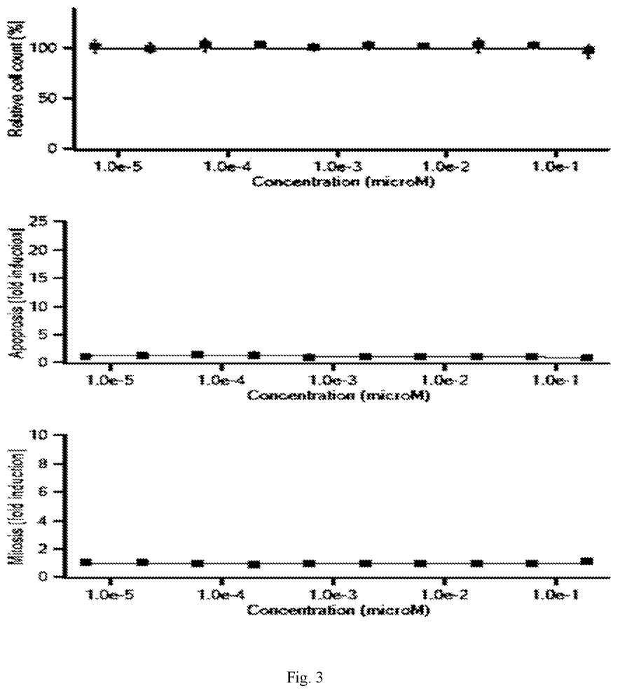

A positive and a negative control experiment were also performed. For a positive control the living MCF7 cells were incubated with Staurosporine for 72 hrs in various concentrations. Staurosporine is a drug that is known in the art to induce apoptosis (FIG. 2). For a negative control a species-matched, non-specific IgG was used to incubate living MCF7 cells for 72 hrs in various concentrations (FIG. 3).

The results clearly show that that incubation of the MCF7 cells with the antibodies of Example 2 can induce apoptosis and this result is concentration dependent. To further characterize the antibodies of example 2 Western Blot analysis was performed.

### Example 4—Humanization of Monoclonal Antibodies

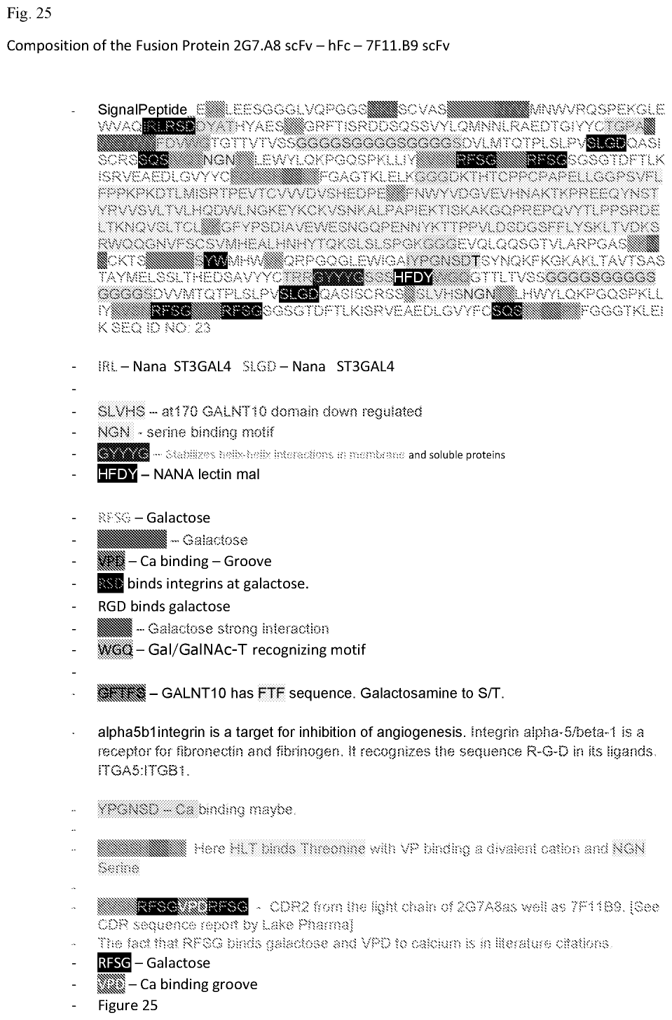

The selected monoclonal antibodies were humanized in accordance with standard procedures, as outlined in FIG. 25.

### Example 5—Generation of Fusion Protein

A fusion protein was created comprising a first binding domain derived from a first humanized monoclonal antibody identified in Example 1, comprising a first variable heavy domain (V_{H1}) and a first variable light domain (V_{L1}) connected by a first linker (L₁) and a second humanized monoclonal antibody identified in Example 1, comprising a second variable heavy domain (V_{H2}) and a second variable light domain (V_{L2}) connected by a second linker (L₂); and a human Fc domain (Fc)

The fusion protein was generated using humanized clone 1 and 2 (2G7.A8 mAb and 7F11.B9) mAb from Example 2.

The Fc fusion protein was constructed by coupling the variable heavy and variable light domains of clone: 7F11.B9; Isotype Ig1 k (shown in SEQ ID NO:1) and the variable heavy and variable light domains of clone 2G7.A8; Isotype Ig1 k (Shown in SEQ ID NO:2) using peptide linkers in combination with an Fc domain.

The Fc portion of the fusion protein was taken from an IgG1. Specifically, amino acid residues 29-255 from the 4CDH_A Chain A, IG gamma-1 chain C region from Homo sapiens.

The specific composition of the resulting Fc Fusion protein is shown in FIG. 25, having the polypeptide sequence of SEQ ID NO:23.

The construct 7F11.B9 scFv-hFc-2G7.A8 was cloned into a high expression mammalian vector (LakePharma's proprietary vector). The vectors were digested and sequenced to confirm the correct sequence was integrated into the expression vector.

HEK293 cells were seeded in shake flask 24 hours before transfection, and were grown using serum-free chemically defined media. The DNA expression construct was transiently transfected into 0.03 L suspension of HEK293 using standard laboratory techniques. After 24 hours, cells were counted to obtain the viability and viable cell count, and titer was measured by ForteBio Octet.

Additional readings were taken throughout the transient transfection production run. The culture was harvest after 5 days.

The conditioned media supernatant was harvested from the transient transfection production run by centrifugal spinning. The protein was purified by Protein A chromatography. Filtration using a 0.2 μm membrane filter was performed. After purification and filtration, 0.47 mg of protein was obtained. SDS-PAGE analysis was performed and the gel was stained with SimplyBlue SafeStain solution to confirm protein expression.

### Example 6—Apoptotic Activity of Fusion Protein on Cancerous Cells

Further apoptosis assays were performed to determine if the fusion protein could cause apoptosis in cancer cell lines. Apoptosis was determined by microscopy, wherein a kit was used (Abcam Apoptosis/Necrosis Detection Kit ab176749) and protocol followed according to the manufacturer's guidelines. Cells were grown in RPMI1640, 10% FBS, 2 mM L-alanyl-L-Glutamine, 1 mM Na Pyruvate or a special medium. Cells were seeded into 384-well plates and incubated in a humidified atmosphere of 5% CO2 at 37° C. The fusion protein was added the day following cell seeding at a concentration of 8.3 μM. At the same time, a time zero untreated cell plate was generated. After an 8 day incubation period, cells were fixed and stained to allow fluorescence visualization of nuclei. At 5 days post seeding, the growth media was replaced and the plates were re-dosed with the test agent.

The test agent was diluted 10-fold from the stock sample as specified in the above table, and assayed in triplicate. Automated fluorescence microscopy was carried out using a Molecular Devices ImageXpress Micro XL high-content imager, and images were collected with a 4× objective. 16-bit TIFF images were acquired and analyzed with MetaXpress 5.1.0.41 software. Cell proliferation was measured by the fluorescence intensity of an incorporated nuclear dye. The output is referred to as the relative cell count, where the measured nuclear intensity is transformed to percent of control (POC) using the following formula:

$$POC = \frac{lx}{lo} \times 100$$

Where Ix is the nuclear intensity at concentration x, and lo is the average nuclear intensity of the untreated vehicle wells.

Time zero non-treated plate was used to determine the number of doublings during the assay period, using the formula:

$$Doublings = log 2 ( \frac{N}{NTo} )$$

Where N is the cell number in untreated wells at the assay end point and NTO is the cell number at the time of compound addition.

An antibody to activated caspase-3 was used to label cells from early to late stage apoptosis (Ref.3). The output is shown as a fold increase of apoptotic cells over vehicle background normalized to the relative cell count in each well.

### Criteria for Positive Responses:

P-value<0.05 when comparing treated to untreated wells.
Apoptosis: >5-fold increase above mean of vehicle background in activated caspase-3 signal indicates an apoptotic response

TABLE 2
Summary for untreated cells

| Plate | | Relative cell count | | | Apoptosis (fold induction) | | | |
|---|---|---|---|---|---|---|---|---|
| # | Cell line | Mean | StdDev | CV | Mean | StdDev | CV | Doublings |
| 1 | BPH1 | 100 | 5.89 | 0.06 | 1.00 | 0.18 | 0.18 | 5.32 |
| 1 | Hs 294T | 100 | 2.63 | 0.03 | 1.00 | 0.06 | 0.06 | 4.19 |
| 1 | SW948 | 100 | 11.28 | 0.11 | 1.00 | 0.26 | 0.26 | 4.29 |
| 1 | CaOV3 | 100 | 13.90 | 0.14 | 1.00 | 0.26 | 0.26 | 3.09 |
| 1 | CFPAC-1 | 100 | 15.89 | 0.16 | 1.00 | 0.17 | 0.17 | 2.85 |
| 1 | 5637 | 100 | 9.97 | 0.10 | 1.00 | 0.23 | 0.23 | 4.05 |
| 1 | C-4 II | 100 | 17.82 | 0.18 | 1.00 | 0.23 | 0.23 | 3.96 |
| 1 | SNU-423 | 100 | 9.31 | 0.09 | 1.00 | 0.14 | 0.14 | 3.42 |
| 1 | Detroit 562 | 100 | 3.66 | 0.04 | 1.00 | 0.06 | 0.06 | 4.31 |
| 1 | U-138MG | 100 | 10.58 | 0.11 | 1.00 | 0.13 | 0.13 | 2.46 |

TABLE 3
Summary table for treated wells (Italics indicates a significant response)

| Plate | | Relative cell count | | | Apoptosis (fold induction) | | | |
|---|---|---|---|---|---|---|---|---|
| # | Cell line | Mean | StdDev | CV | Mean | StdDev | CV | Doublings |
| 1 | BPH1 | 18.48 | 1.47 | 0.08 | 8.49 | 1.33 | 0.16 | 2.88 |
| 1 | Hs 294T | 20.56 | 8.77 | 0.43 | 16.08 | 3.72 | 0.23 | 1.91 |
| 1 | SW948 | 69.09 | 17.70 | 0.26 | 2.62 | 0.64 | 0.25 | 3.76 |
| 1 | CaOV3 | 62.60 | 5.32 | 0.08 | 2.12 | 0.27 | 0.13 | 2.41 |
| 1 | CFPAC-1 | 127.04 | 34.75 | 0.27 | 0.67 | 0.38 | 0.57 | 3.20 |
| 1 | 5637 | 14.43 | 5.63 | 0.39 | 9.29 | 2.92 | 0.31 | 1.25 |
| 1 | C-4 II | 2.54 | 1.76 | 0.69 | 38.04 | 24.22 | 0.64 | −1.34 |
| 1 | SNU-423 | 14.05 | 6.46 | 0.46 | 10.33 | 5.68 | 0.55 | 0.59 |
| 1 | Detroit 562 | 31.53 | 6.88 | 0.22 | 3.58 | 0.47 | 0.13 | 2.65 |
| 1 | U-138MG | 73.21 | 8.77 | 0.12 | 2.28 | 0.14 | 0.06 | 2.01 |

After 2 doses of the fusion protein within 7 days levels of apoptosis between 50% to 80% were observed. The only exceptions were in pancreatic cell line, which resisted apoptosis and the glioblastoma cell line which had a level of 20% apoptosis.

Surprisingly, apoptosis was observed in 24 out of the 25 cancer cell lines tested in vitro.

In summary, the fusion protein causes apoptosis in cancer cells but not in normal cell lines.

### Example 7—Apoptotic Activity of Fusion Protein on Non-Cancerous Cells

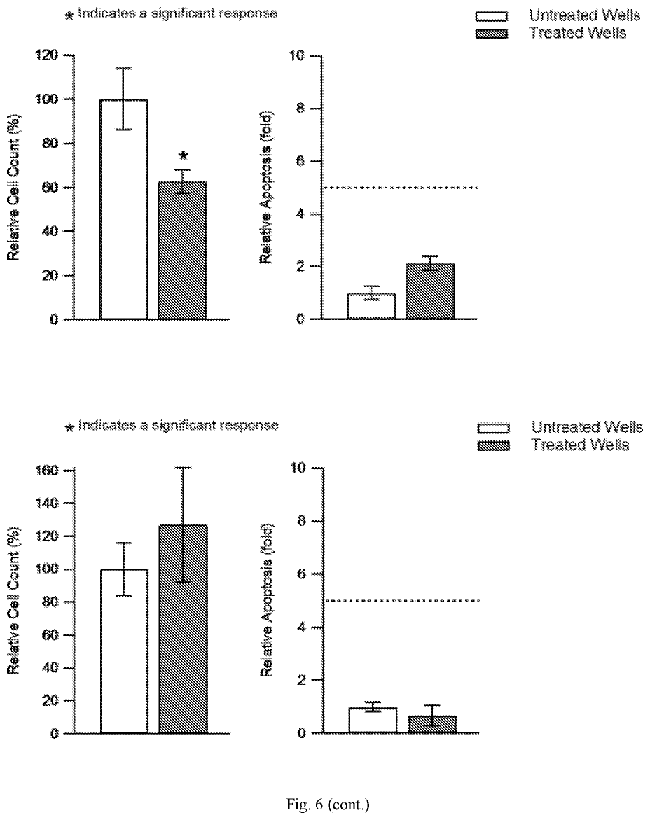

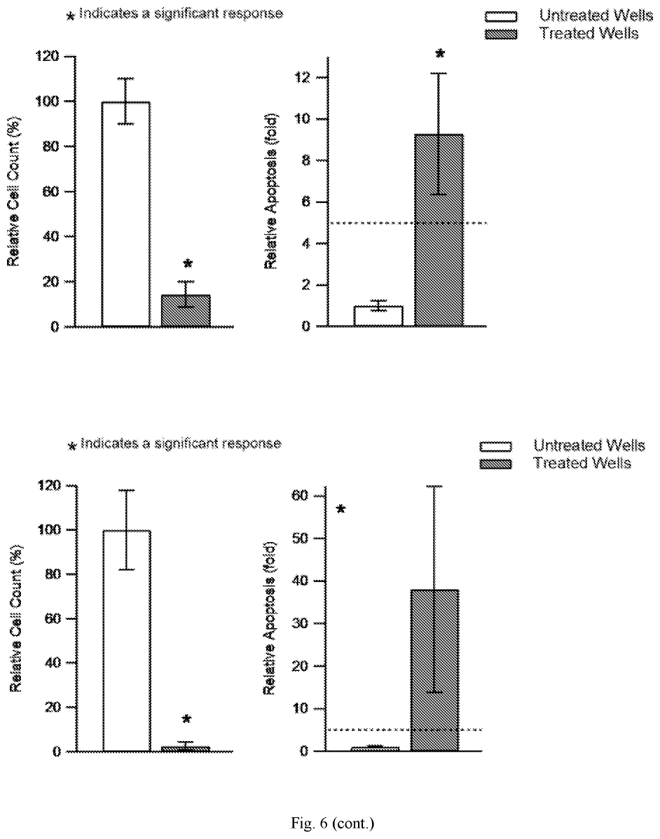

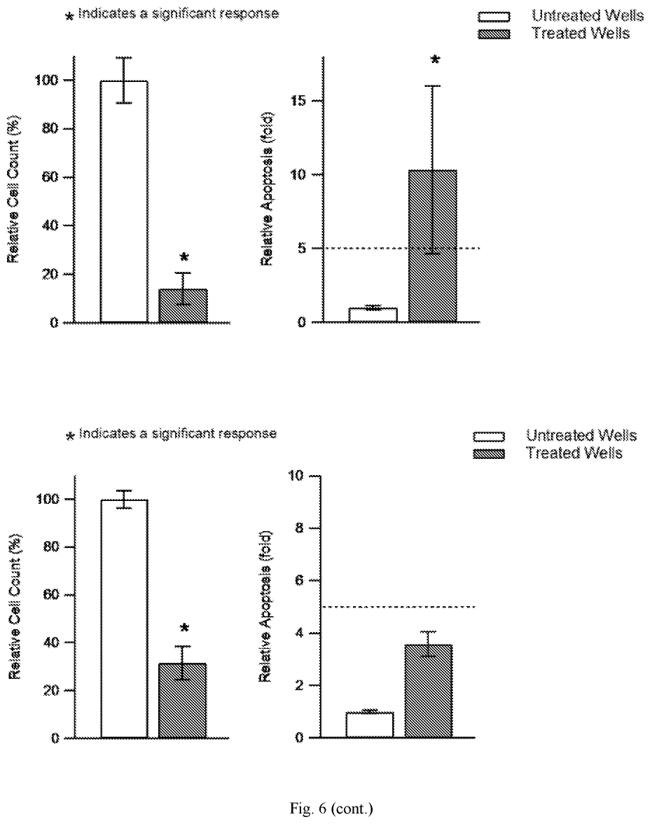

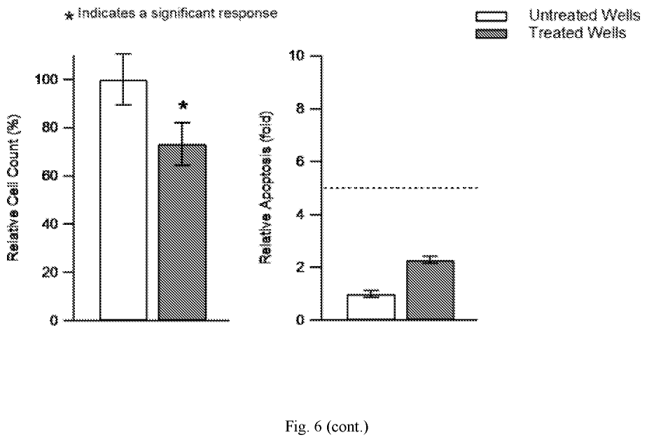

The fusion protein was then tested in non-cancer cell lines: MCF10A (normal breast cancer cell line) and hepatocytes (Thermofisher, HMCS2S). No apoptosis was observed in either cell line. This was in contrast to Staurosporine which yielded high levels of apoptosis in MCF10A cells. (FIGS. 8-11)

For the detection of apoptosis/necrosis an Abcam kit was used (Apoptosis/Necrosis Detection Kit (blue, green, red); product name: ab176749) with adherent cells and suspension cells (from Thermo Fisher).

The Apoptosis/Necrosis Detection Kit (blue, green, red) (ab176749) is designed to simultaneously monitor apoptotic, necrotic and healthy cells. The PS sensor used in this kit has green fluorescence (Ex/Em=490/525 nm) upon binding to membrane PS. Necrosis has been characterized as passive, accidental cell death resulting from environmental perturbations with uncontrolled release of inflammatory cellular contents. Loss of plasma membrane integrity, as demonstrated by the ability of a membrane-impermeable 7-AAD (Ex/Em=546/647 nm) to label the nucleus, represents a straightforward approach to demonstrate late stage apoptosis and necrosis. In addition, this kit also provides a live cell cytoplasm labelling dye, CytoCalcein Violet 450 (Ex/Em=405/450 nm), for labelling living cell cytoplasm. This kit is optimized to simultaneously detect cell apoptosis (green), necrosis (green and/or red) and healthy cells (blue) with a flow cytometer or fluorescence microscope. Notes Apoptosis is an active, programmed process of autonomous cellular dismantling that avoids eliciting inflammation. In apoptosis, phosphatidylserine (PS) is transferred to the outer leaflet of the plasma membrane. As a universal indicator of the initial/intermediate stages of cell apoptosis, the appearance of phosphatidylserine on the cell surface can be detected before morphological changes are observed.

The tested applications for this study were fluorescence microscopy, flow cytometry and functional studies. Fluorescent analysis showing cells that are live (blue, stained by CytoCalcein Violet 450), apoptotic (green, Apopx in Green Indicator), and necrotic (red, indicated by 7-AAD staining).

### Hepatocytes

### Hepatocyte Culturing Protocol

Pipette
Clean bench
Incubator
Centrifuge
Water bath
Microscope Materials
Greiner 10 cm & 96 well plates
BEGM (500 mL basal medium, frozen gentamycin/Amphoterjcin, epinephrine) (add extra 5 ng/mL EGF, 70 ng/mL phosphoethanolamine and 10% FBS) (THERMOFISHER)
Pipette tips
Fibronectin (SIGMA-ALDRICH)
Bovine collagen type I (THERMOFISHER)
Bovine serum albumin (SIGMA-ALDRICH)
Soybean trypsin Inhibitor (SIGMA-ALDRICH)
Trypsin-EDTA (GIBCO)
Trypsin-53 mM EDTA (GIBCO)

### Method

1. Flask coating (done under clean bench)

Prepare a mixture of 0.01 mg/mL fibronectin, 0.03 mg/mL bovine collagen type I, and 0.01 mg/mL bovine serum albumin dissolved in culture medium (BEGM). For growth area of 75 cm², add 4.5 mL of coating solution and rock gently to coat the entire surface. Incubate the freshly coated vessel in a 37° C. incubator overnight (it is preferable to use tissue culture vessels with tightened, plug-seal caps to prevent evaporation during the coating process). Suction off solution before planting cells.

2. Check cells under microscope for confluency before sub culturing. A ratio of 1:3 to 1:6 is recommended every 2 to 3 days.

3. Sub-culturing (done under clean bench, covered when moving out from under the clean bench). Remove and discard culture medium. Briefly rinse the cell layer with 0.05% (w/v) Trypsin-53 mM EDTA solution to remove all traces of serum which contains trypsin inhibitor. Add 2.0 to 3.0 ml of trypsin-EDTA solution to flask/plate and observe cells under an inverted microscope until cell layer is dispersed (usually 5 to 15 minutes). Note: to avoid clumping do not agitate cells by hitting or shaking the flask while waiting for the cells to detach. Cells that are difficult to detach may be placed at 37° C. to facilitate dispersal. Add 0.1% Soybean Trypsin Inhibitor and aspirate cells by gently pipetting.

To remove trypsin-EDTA solution, transfer cell suspension to centrifuge tube and spin approximately at 125× g for 5 to 10 minutes. Discard supernatant and resuspend cells in fresh growth medium. Add appropriate aliquots of cell suspension to new coated culture vessels. Place culture vessels in incubators at 37° C.

The results indicated that no apoptosis was observed after 48 hours.

### Example 8—Toxicity

The individual scfv sequences are highly toxic to cells. However, the fusion protein is not toxic at a concentration of 4.1 μM to normal cells.

Furthermore, zebra fish embryo toxicity tests were conducted over a 14 day period. Zebra fish embryos were injected with the fusion protein at twice the concentration of the therapeutic dose on day 1, day 3, day 7, and day 10. Observations were recorded for acute toxicity, hepatoxicity, ototoxicity and cardiotoxicity. Behavioral assays were also conducted.

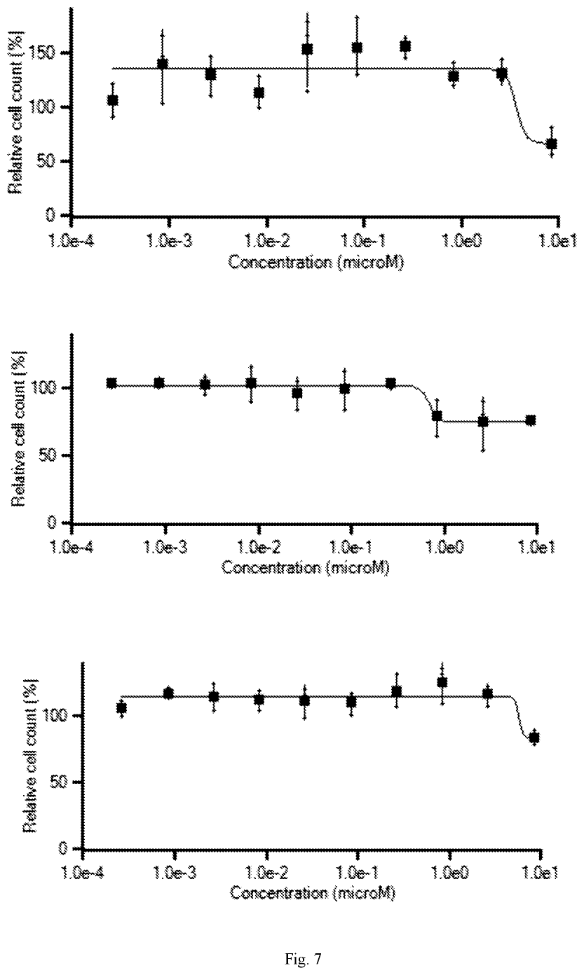

After 14 days no toxicity was observed in the zebra fish embryos. (FIG. 12)

### Example 9—Sequestration in Lysosomes

Microscopy also revealed that the fusion protein was sequestered only in the lysosome. The fusion protein was labelled with 488a dye (Thermo Fisher), which is green. The lysosomes were stained red. Co-localisation was observed by colour combination where Green combined with red yielded yellow.

The localization to the lysosome was observed to be rapid, in bladder cancer cell lines the localization took place in less than 15 seconds. Moreover, according to microscopy 100% of the fusion protein was sequestered to the lysosome.

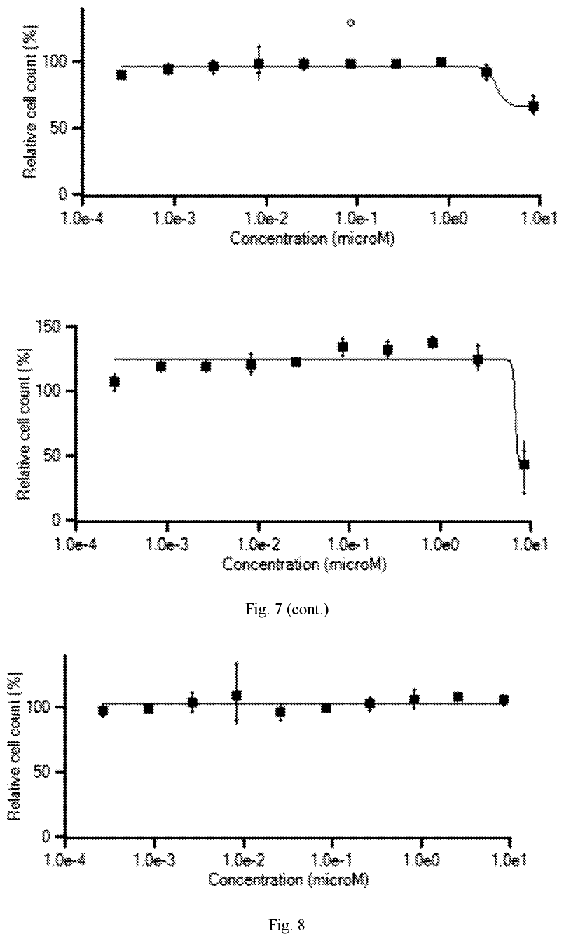

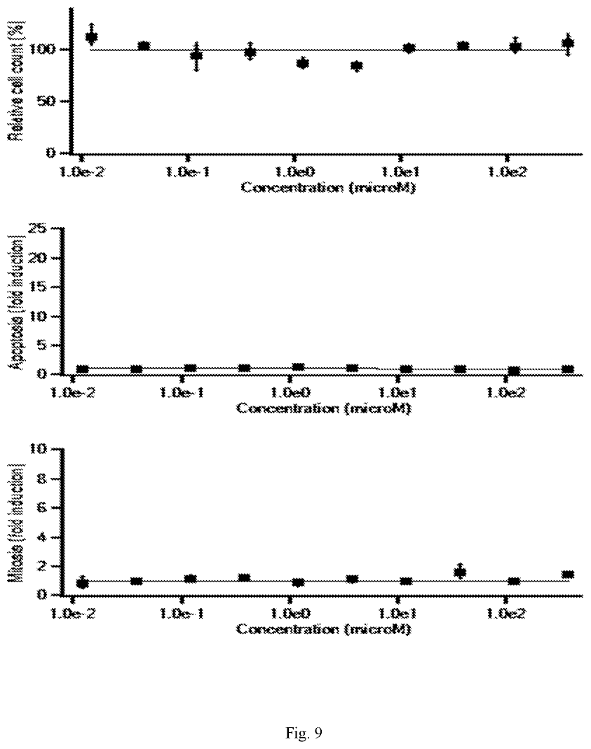

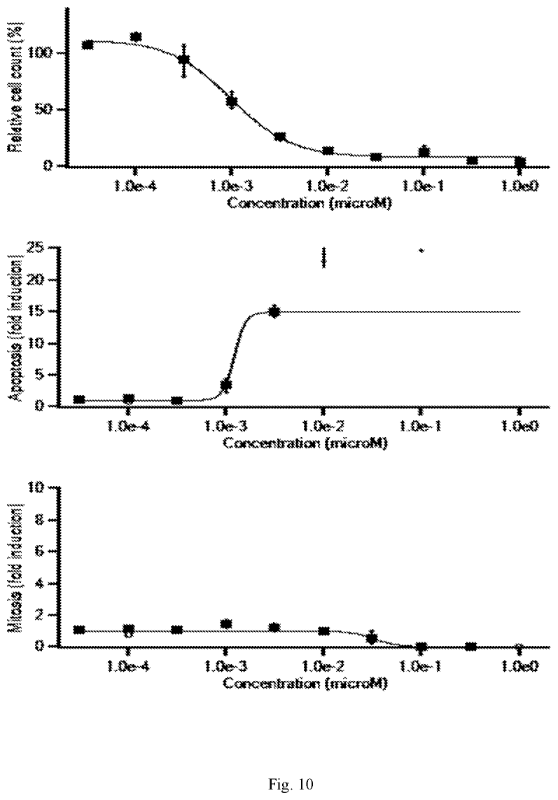

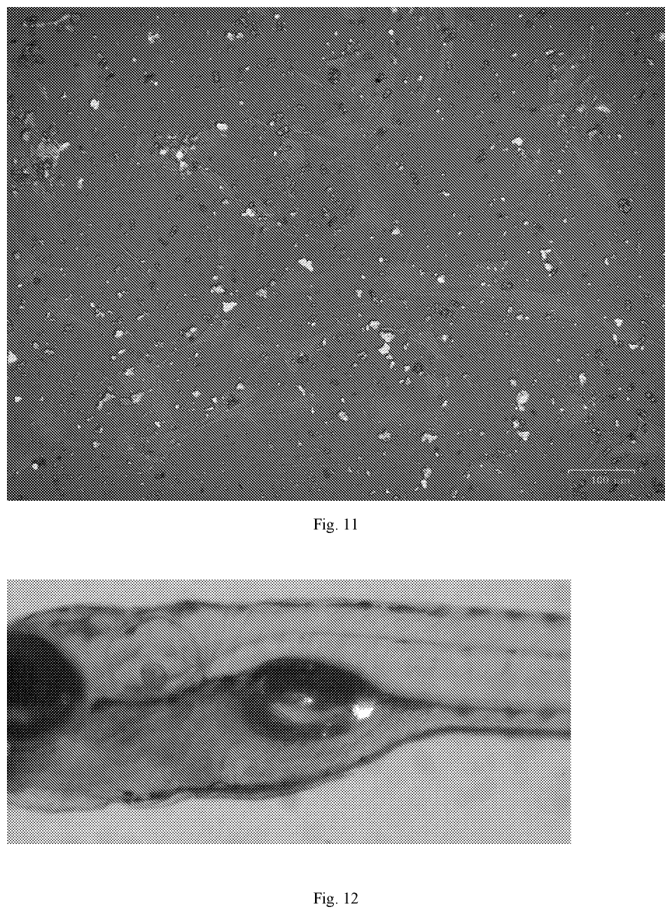

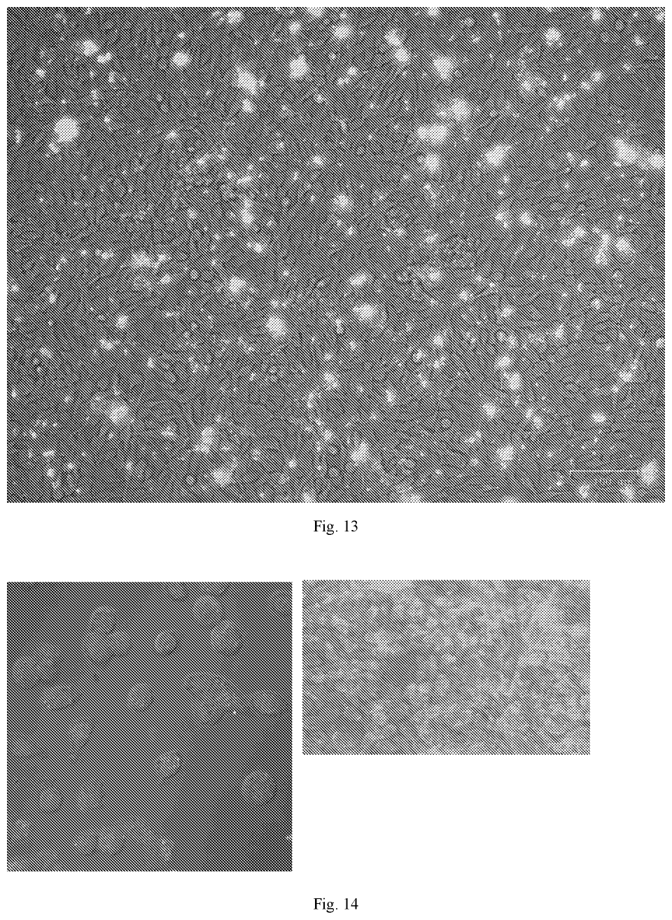

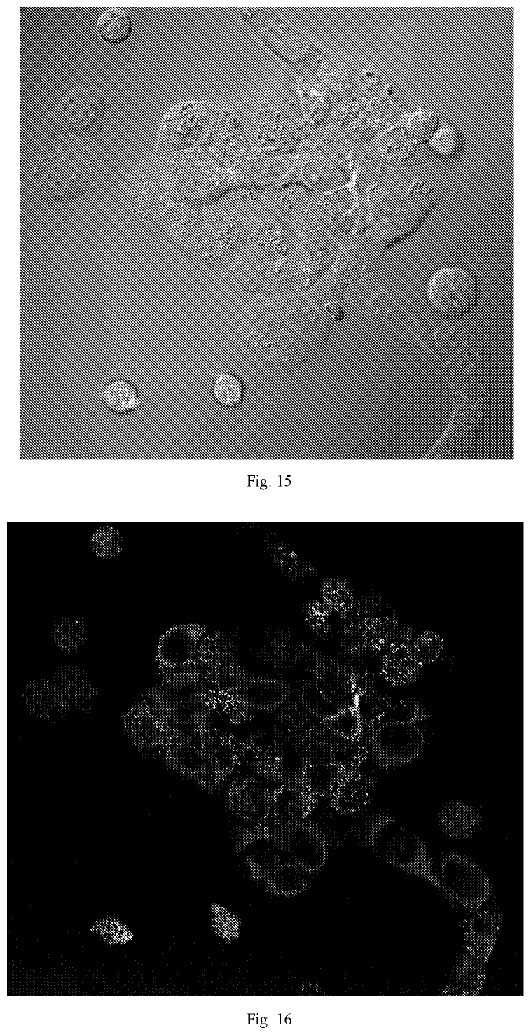

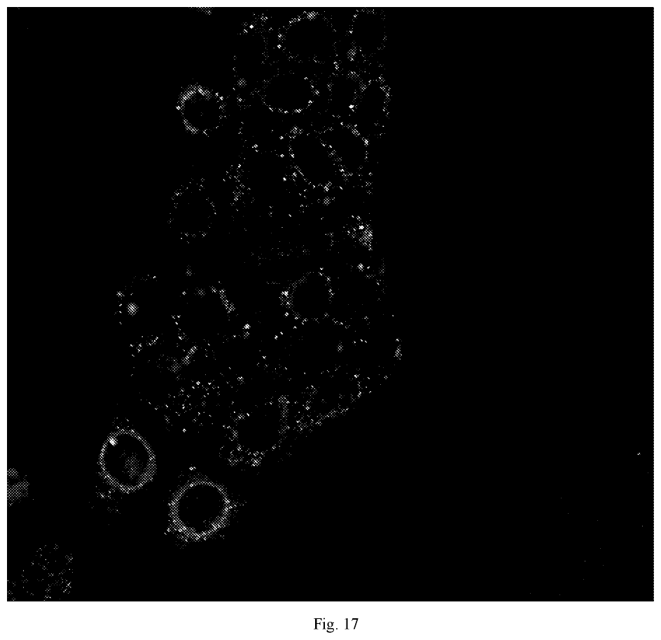

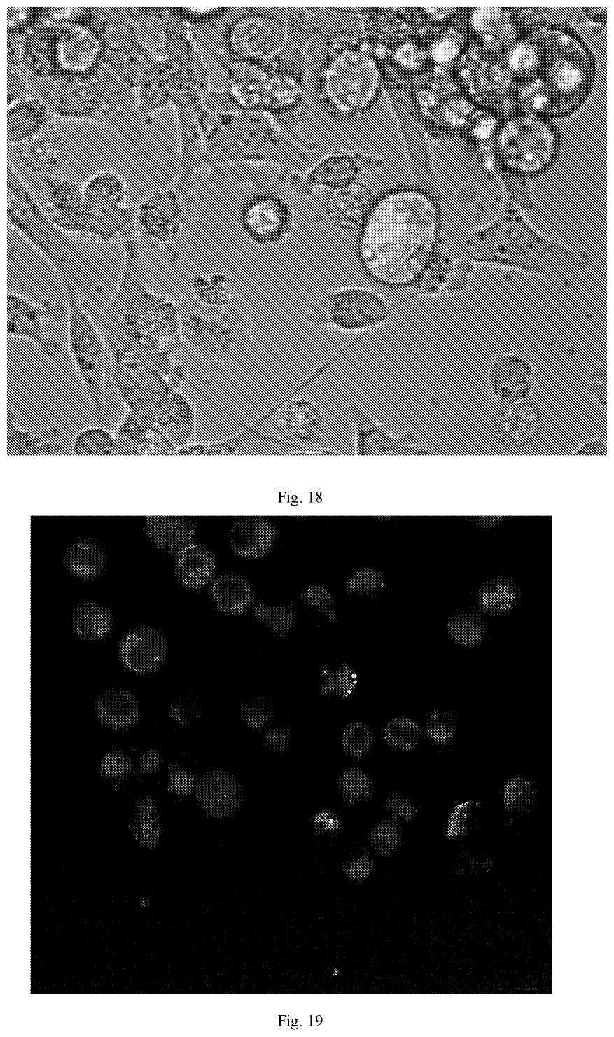

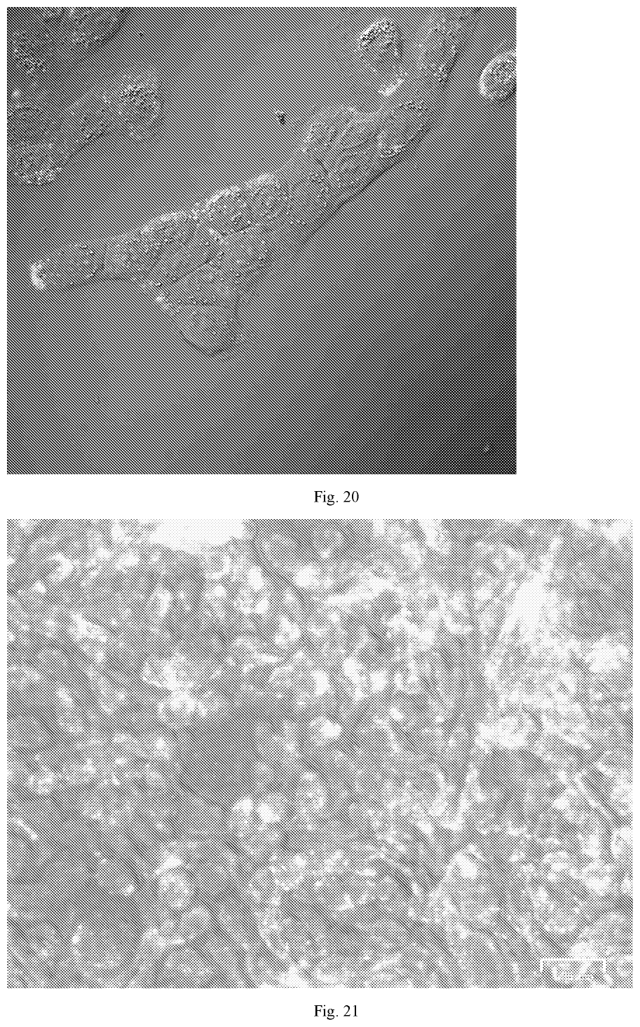

In contrast, co-localisation was not observed for other organelles such as nucleus, ribosomes, mitochondria, ER or the cell membrane. (FIG. 13-FIG. 21)

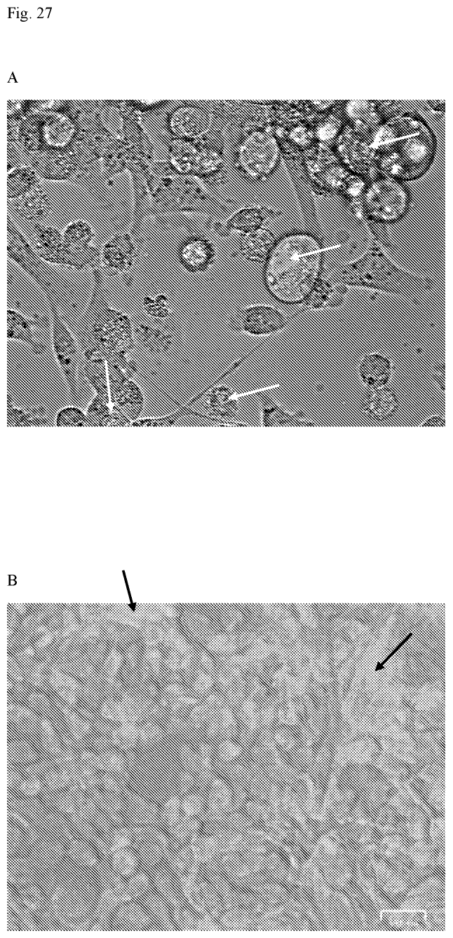

Additionally, rapid localization of the fusion protein to the lysosome was observed in triple negative breast cancer cell lines, as shown in FIG. 27 A and B.

Without wishing to be bound to a particular theory, it appears that the sequestration to the lysosome leads to natural cell death of the cancer cells.

### Example 10—Apoptotic Mechanism of Action

Mass spectrometry was performed on treated vs non-treated cells in order to further understand the underlying mechanism by which the fusion protein is able to cause apoptosis in cancer cells.

Occupancy of O-glycosylation sites can vary in-vivo depending on the cells that are expressing the protein. Steentoft C, “Precision mapping of the human O-GalNAc glycoproteome through SimpleCell technology”, EMBO J, 32(10):1478-88, May 15, 2013.

The active sites of the proteins were determined using a protein painting protocol.

### Site Prediction for O-Glycosylation

Pro at +3 and/or −1 positions from T/S strongly favours glycosylation irrespective of single and multiple glycosylation sites. In addition, serine and threonine are preferred around the multiple glycosylation sites due to the effect of clusters of closely spaced glycosylated Ser/Thr

### Equipment Used:

Clean Bench, CO₂ incubator, Centrifuge, Pipettes, Autoclave, Sonicator, Vortexer

### Materials Used:

10 cm cell dishes, Pipette tips, PBS solution, Antibody, Cell culture medium, Chloroform, Centrifuge Tubes, prepared protein paint mix

Protein Paint Contains: All paints purchased through MilliPore-Sigma (Sigma-Aldrich)

### Paint #1)

### 4-AMINO-3,5-DI-ME-1-(4-ME-BZL)-4H-(1,2,4)TRIAZOL-1-IUM, NAPHTHALENE-2-SULFONATE

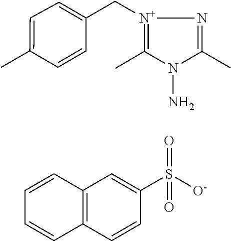

### Paint #2)

### Sodium 4-(4-(BENZYL-ET-AMINO)-PH-AZO)-2,5-DI-CL-BENZENESULFONATE (Acid Orange 50)

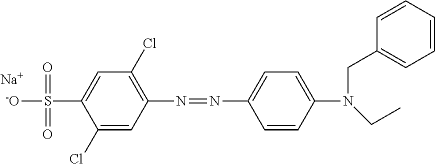

### Paint #3)

Acid Red 17 (Newport Acid Bordeaux B Conc. C.I. 16180)

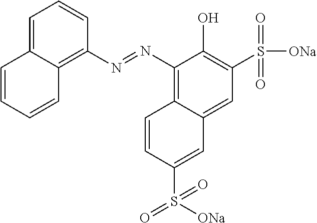

### Protein Paint Mixing:

Paints were weighed and ready before starting extraction and painting protocol.

Paints were dissolved within a solution containing an optimal amount of sample an excess of 1000M to reach a final volume 50 mL of PBS. Alternatively, the paints were prepared before starting protocol by preparing 50 mL of PBS with an excess of 1000M of all three paints, then applied to the sample accordingly and following rest of procedure.

### Procedure:

2) Culture cell lines in 10 cm dishes.
3) Treat the cells with the Antibody at a concentration of 6.5 mg/mL. Allow 72 hrs of treatment.
4) Pull off medium and wash cells with PBS. Use TrypLE Express to pull off adherent cells.
5) Count the number of cells using a hemocytometer or cell counting instrument
6) Add a small amount of fresh medium and pellet cells in a 15 mL centrifuge tube to remove the TrypLE Express. Wash with PBS.
7) Add 100 μL of cold RIPA lysis buffer for each 10⁶ cells. Let the reaction run for 30 minutes on ice, vortexing occasionally.
8) Sonicate the sample for 1 min to break up the cells. Keep sample on ice during this time.
9) Centrifuge at 10,000×g for 20 minutes at 4° C. to pellet cell debris.
10) Transfer supernatant, containing the proteins to a fresh microfuge tube containing a solution of 2:1.8:2 MeOH:DI:Chloroform without disturbing the pellet.
11) Vortex new solution, then let sit for 3-5 minutes. Repeat twice.
12) Centrifuge to pellet cell debris and to separate the two layers.
13) Pull off organic layer and store in −20° C. for analysis.
14) To the aqueous fraction, add solution of paints in PBS at a 1000M excess
15) Vortex solution, and let interact for 5 minutes
16) Add solution to Nanosep tubes and spin to remove excess paints. Wash with PBS
17) Add 2M Urea to the solution to break the protein target complex.
18) Add 10 mM DTT and let react for 15 minutes at 37° C.
19) Alkylate the complex with 50 mM of Iodoacetamide (15 minutes at RT)
20) Digest with Trypsin at a 1:10 protease:protein ratio for 2 hours at 37° C.
21) Add 6 mL of acetic acid to stop reaction
22) Use Zip-Tip to purify the cut protein fragments, following the manufacturer's instructions

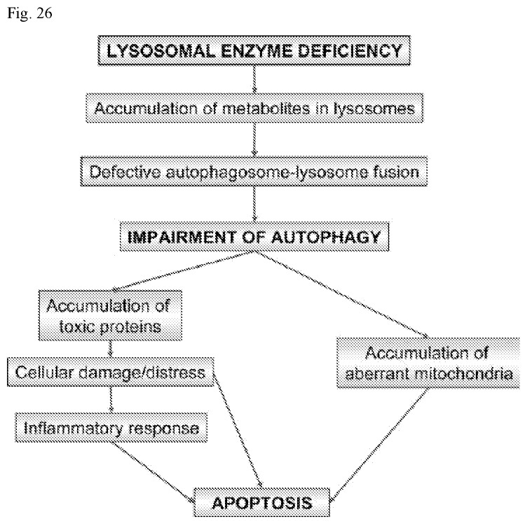

Mass spectrometry analysis in prostate and bladder cancer cell lines identified that many heat-shock proteins were targeted by the fusion protein. Without wishing to be bound to a particular theory, targeting of heat shock proteins is thought to induce autophagy via caspase 3. The process via which autophagy & apoptosis occurs via a Caspase 3 mechanism is outlined in FIG. 26.

Other targets that were bound to the fusion protein were proteins expressed by the genes: ACTB; HSPD1; LMNA; HMGB1; HNRNPA2B1; HSPA5; DLD; P4HB; CKAP4; HIST1H1E; HIST1H1D; HIST1H1C; and HIST1H1B

ATG4B was also upregulated in bladder and prostate cancer cell lines in fusion protein treated cells versus untreated cancer cell lines.

WDR92 was upregulated when HIST1 was targeted by the fusion protein in bladder and prostate cancer cell lines which is thought to lead to an apoptosis cascade.

SMYD5 was also upregulated from metabolomic profile of bladder and prostate cell lines.

The cancer epitope—Sialylation coupled with O-glycosylation is the defining moment of cancer. O-glycosylation with the transfer of GalNAc to serine and threonine residues on proteins by a family of UDP-GalNAc:polypeptide N-acetylgalactosaminlytransferases is the defining moment in the process of oncogenesis. The mass spectroscopic data revealed that the enzyme, ST3Gal-1 is downregulated on treatment of bladder and prostate cancer cell lines with the fusion protein. Alpha-2,3-sialyltransferase (ST3Gal-I) the MOA major path to carcinogenesis is downregulated. The fusion protein developed targets ST3Gal-1 in cancerous cells for destruction with minimal or manageable collateral damage to healthy cells. Thus, the sialylation step is inhibited. The sialylation step is a major cancer pathway.

Finally, the analysis indicated that P2X7 was not bound by the fusion protein.

### Example 11

RightZAP1.3 DNA is a 29.0 kb linear DNA fragment that encompasses bp 3504-right end of the Ad5 genome. The E3 region is deleted (2.7 kb). The fiber protein is a hybrid Ad5/35. It contains the N-terminal tail of Ad5 fused to the shaft and knob of Ad35. The DNA is purified from a cosmid. It is used in combination with shuttle vectors pZAP1.1 or pZAP1.2 to construct replication-deficient adenoviruses containing transgenes (nucleic acid encoding the fusion protein) in place of the E1 region of the Ad5 genome, and the fiber shaft and knob from Ad35. The maximum transgene capacity of the resulting virus is 8.3 kb.

Ad35 belongs to the adenovirus subgroup B that uses almost exclusively CD46 as the primary attachment receptor. CD46 is a membrane protein that is expressed ubiquitously, has complement regulatory functions, and is upregulated in tumor and stem cells.

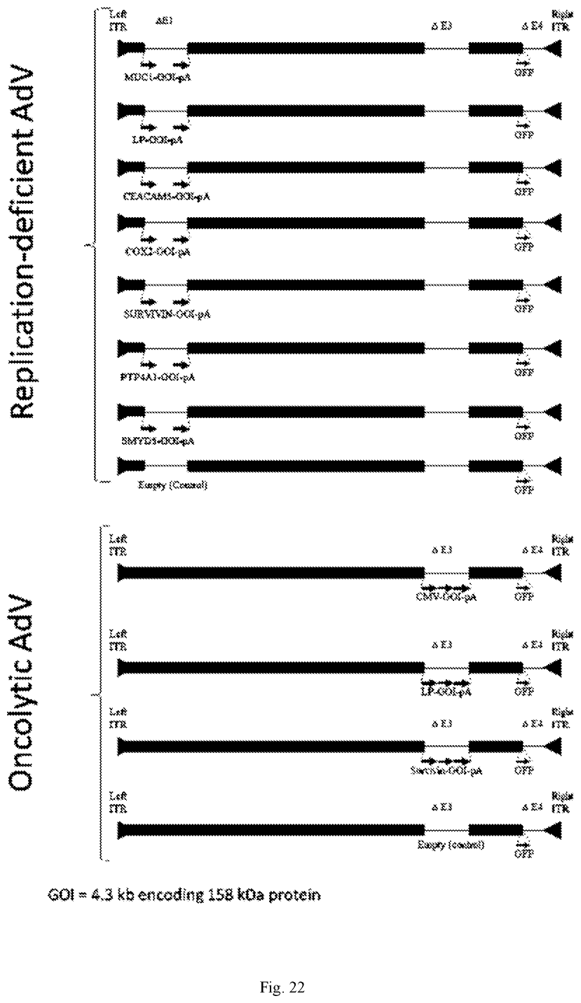

pAd1129-27 is a shuttle plasmid designed for constructing adenovirus vectors characterized by a 2.7 kb deletion in the E3 region, and a hybrid Ad5/3 fiber. A 2.7 kb BglII fragment including E3 6.7K, gp19K membrane protein, the adenovirus “death” protein ADP, RID-a, RID-ß, and 14.7K is deleted, and replaced with a multiple cloning site. The E3 12.5K ORF is truncated. The U exon is intact. The Ad5/3 hybrid fiber is made of the N-terminal tail and shaft of Ad5 fused to the knob of Ad3. pAd1129-27 is used to construct a replication-deficient or oncolytic adenovirus vector expressing large transgenes (inserted into the E3 region itself or elsewhere), or multiple expression cassettes (for instance two independent expression cassettes, one in the E1 region, and the other in the E3 region). Expression cassettes inserted into the E3 region contain a promoter and poly(A) signal, but no intron nor splice site. The adenovirus sequences present in pAd1129-27 is flanked by two SfiI sites, which generate non-symmetrical sticky ends suitable for directional cloning with the other AdenoQuick2.0 plasmids (pAd1127, pAd1128, pAd1130, and their derivatives). (FIG. 22)

Specifically, eight promoter switches will work efficiently in cancer cells and not in normal cells. Each of the eight promoter sequences are known in the art.

SEQUENCES

SEQ ID NO: 1 Clone 6H5.C10; Isotype Ig1 k V_{H}
QVTLKESGPGILQPSQTLSLTCSFSGFSLSTFGMGVGWIRQPSGKGLEWLAHIWWDDYK
YYNPALKSRLTISKDTSKNQVFLKIANVDTADTATYYCARISYYGSSYLDYWGQGTTLT
VSS
SEQ ID NO: 2 Clone 6H5.C10; Isotype Ig1 k V_{L}
DVVMTQTPLSLPVSLGDQASISCRSSQSLVHSNGNTYLHWYLQKPGQSPKLLIYKVSNR
FSGVPDRFSGSGSGTDFTLKISRVEAEDLGVYFCSQTTHVPYTFGGGTKLEIKR
SEQ ID NO: 3 Kazald1 promoter
ggtttggctggcgggcccctaggaatgctctcctccccgcccccggcccAAAGTGCCACG
SEQ ID NO: 4 VH1 CDR1
GFTFSNYW
SEQ ID NO: 5 VH1 CDR2
IRLRSDDYAT
SEQ ID NO: 6 VH1 CDR3
TGPANWGYWYFDV
SEQ ID NO: 7 VL1 CDR1
QSIVQTNGNTY
SEQ ID NO: 8 VL1 CDR2
KVS
SEQ ID NO: 9 VL1 CDR3
FQGSHVPLT
SEQ ID NO: 10 VH2 CDR1
GYTFTSYW
SEQ ID NO: 11 VH2 CDR2
IYPGNSDT
SEQ ID NO: 12 VH2 CDR3
TRRGYYYGSSSHFDY
SEQ ID NO: 13 VL2 CDR1
QSLVHSNGNTY
SEQ ID NO: 14 VL2 CDR2
KVS
SEQ ID NO: 15 VL2 CDR3
SQSTHVPPT
SEQ ID NO: 16 VH1
EVKLEESGGGLVQPGGSMKLSCVASGFTFSNYWMNWVRQSPEKGLEWVAQIRLRSDD
YATHYAESVKGRFTISRDDSQSSVYLQMNNLRAEDTGIYYCTGPANWGYWYFDVWGT
GTTVTVSS
SEQ ID NO: 17 VL1
DVLMTQTPLSLPVSLGDQASISCRSSQSIVQTNGNTYLEWYLQKPGQSPKLLIYKVSNRF
SGVPDRFSGSGSGTDFTLKISRVEAEDLGVYYCFQGSHVPLTFGAGTKLELK
SEQ ID NO: 18 VH2
EVQLQQSGTVLARPGASVKMSCKTSGYTFTSYWMHWVKQRPGQGLEWIGAIYPGNSD
TSYNQKFKGKAKLTAVTSASTAYMELSSLTHEDSAVYYCTRRGYYYGSSSHFDYWGQ
GTTLTVSS
SEQ ID NO: 19 VL2
DVVMTQTPLSLPVSLGDQASISCRSSQSLVHSNGNTYLHWYLQKPGQSPKLLIYKVSNR
FSGVPDRFSGSGSGTDFTLKISRVEAEDLGVYFCSQSTHVPPTFGGGTKLEIK
SEQ ID NO: 20 L1 AND L2
GGGGSGGGGSGGGG
SEQ ID NO: 21 L3 AND L4
GGG
SEQ ID NO: 22 HUMAN FC
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGK
SEQ ID NO: 23 FUSION PROTEIN FULL LENGTH
EVKLEESGGGLVQPGGSMKLSCVASGFTFSNYWMNWVRQSPEKGLEWVAQIRLRSDD
YATHYAESVKGRFTISRDDSQSSVYLQMNNLRAEDTGIYYCTGPANWGYWYFDVWGT
GTTVTVSSGGGGSGGGGSGGGGSDVLMTQTPLSLPVSLGDQASISCRSSQSIVQINGNT
YLEWYLQKPGQSPKLLIYKVSNRFSGVPDRFSGSGSGTDFTLKISRVEAEDLGVYYCFQ
GSHVPLTFGAGTKLELKGGGDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHEALHN
HYTQKSLSLSPGKGGGEVQLQQSGTVLARPGASVKMSCKTSGYTFTSYWMHWVKQRP
GQGLEWIGAIYPGNSDTSYNQKFKGKAKLTAVTSASTAYMELSSLTHEDSAVYYCTRR
GYYYGSSSHFDYWGQGTTLTVSSGGGGSGGGGSGGGGSDVVMTQTPLSLPVSLGDQAS
ISCRSSQSLVHSNGNTYLHWYLQKPGQSPKLLIYKVSNRFSGVPDRFSGSGSGTDFTLKIS
RVEAEDLGVYFCSQSTHVPPTFGGGTKLEIK
SEQ ID NO: 24 NUCLEOTIDE SEQUENCE OF FUSION PROTEIN
GAAGTGAAGCTTGAGGAGTCTGGAGGAGGCTTGGTGCAACCTGGAGGATCCATGAA
ACTCTCCTGTGTTGCCTCTGGATTCACTTTCAGTAACTACTGGATGAACTGGGTCCGC
CAGTCTCCAGAGAAGGGGCTTGAGTGGGTTGCTCAAATTAGATTGAGATCTGATGAT
TATGCAACACATTATGCGGAGTCTGTGAAAGGGAGGTTCACCATCTCAAGAGATGA
TTCCCAAAGTAGTGTCTACCTGCAAATGAACAACTTAAGGGCTGAAGACACTGGAA
TTTATTACTGCACAGGCCCCGCTAACTGGGGTTACTGGTACTTCGATGTCTGGGGCA
CAGGGACCACGGTCACCGTCTCCTCAGGTGGCGGAGGATCTGGCGGAGGCGGTAGT
GGCGGTGGCGGATCTGATGTTTTGATGACCCAAACTCCACTCTCCCTGCCTGTCAGT
CTTGGAGATCAAGCCTCCATCTCTTGCAGATCTAGTCAGAGCATTGTACAGACTAAT
GGAAACACCTATTTAGAATGGTACCTGCAGAAACCAGGCCAGTCTCCAAAGCTCCT
GATCTACAAAGTTTCCAACCGATTTTCTGGGGTCCCAGACAGGTTCAGTGGCAGTGG
ATCAGGGACAGATTTCACACTCAAGATCAGTAGAGTGGAGGCTGAGGATCTGGGAG
TTTATTACTGCTTTCAAGGTTCACATGTTCCGCTCACGTTCGGTGCTGGGACCAAGCT
GGAGCTGAAAGGCGGTGGAGACAAAACTCACACATGCCCACCGTGCCCAGCACCTG
AACTCCTGGGGGGACCGTCAGTCTTCCTCTTCCCCCCAAAACCCAAGGACACCCTCA
TGATCTCCCGGACCCCTGAGGTCACATGCGTGGTGGTGGACGTGAGCCACGAAGAC
CCTGAGGTCAAGTTCAACTGGTACGTGGACGGCGTGGAGGTGCATAATGCCAAGAC
AAAGCCGCGGGAGGAGCAGTACAACAGCACGTACCGTGTGGTCAGCGTCCTCACCG
TCCTGCACCAGGACTGGCTGAATGGCAAGGAGTACAAGTGCAAGGTCTCCAACAAA
GCCCTCCCAGCCCCCATCGAGAAAACCATCTCCAAAGCCAAAGGGCAGCCCCGAGA
ACCACAGGTGTACACCCTGCCCCCATCCCGGGATGAGCTGACCAAGAACCAGGTCA
GCCTGACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGCAGCAGG
GGAACGTCTTCTCATGCTCCGTGATGCATGAGGCTCTGCACAACCACTACACGCAGA
AGAGCCTCTCCCTGTCTCCGGGTAAAGGCGGAGGTGAGGTTCAGCTCCAGCAGTCTG
GGACTGTGCTGGCAAGGCCTGGGGCTTCAGTGAAGATGTCCTGCAAGACTTCTGGCT
ACACATTTACCAGCTACTGGATGCACTGGGTAAAACAGAGGCCTGGACAGGGTCTG
GAATGGATAGGGGCTATTTATCCTGGAAATAGTGATACTAGCTACAACCAGAAGTT
CAAGGGCAAGGCCAAACTGACTGCAGTCACATCCGCCAGCACTGCCTACATGGAGC
TCAGCAGCCTGACACATGAGGACTCTGCGGTCTATTACTGTACAAGAAGGGGCTATT
ACTACGGTAGTAGCTCCCACTTTGACTACTGGGGCCAAGGCACCACTCTCACAGTCT
CCTCAGGTGGCGGAGGATCTGGCGGAGGCGGTAGTGGCGGTGGCGGATCTGATGTT
GTGATGACCCAAACTCCACTCTCCCTGCCTGTCAGTCTTGGAGATCAAGCCTCCATC
TCTTGCAGATCTAGTCAGAGCCTTGTACACAGTAATGGAAACACCTATTTACATTGG
TACCTGCAGAAGCCAGGCCAGTCTCCAAAGCTCCTGATCTACAAAGTTTCCAACCGA
TTTTCTGGGGTCCCAGACAGGTTCAGTGGCAGTGGATCAGGGACAGATTTCACACTC
AAGATCAGCAGAGTGGAGGCTGAGGATCTGGGAGTTTATTTCTGCTCTCAAAGTAC
ACATGTTCCTCCGACGTTCGGTGGAGGCACCAAGCTGGAAATCAAATGA
SEQ ID NO: 25 MUC1 promoter
aCTAGtGTTCATCGGAGCCCAGGTTTACTCCCTTAAGTGGAAATTTCTTCCCCCACTCCCTCCTTGGCTTTCTCCAAG
GAGGGAACCC
AGGCTACTGGAAAGTCCGGCTGGGGGGGGACTGTGGGTTTCAGGGTAGAACTGCGTGTGGAACGGGACAGGG
AGCGGTTAGAAGGGTGGGGCTATTCCG
GGAAGTGGTGGGGGGAGGGAGCCCAAAACTAGCACCTAGTCCACTCATTATCCAGCCCTCTTATTTCTCGGCCCC
GCTCTGCTTCAGTGGACCCGGGGAG
GGCGGGGAAGTGGAGTGGGAGACCTAGGGGTGGGCTTCCCGACCTTGCTGTACAGGACCTCGACCTAGCTGGCT
TTGTTCCCCATCCCCACGTTAGTTGT
TGCCCTGAGGCTAAAACTAGAGCCCAGGGGCCCCAAGTTCCAGACTGCCCCTCCCCCCTCCCCCGGAGCCAGGGA
GTGGTTGGTGAAAGGGGGAGGCCAG
CTGGAGAACAAACGGGTAGTCAGGGGGTTGAGCGATTAGAGCCCTTGTACCCTACCCAGGAATGGTTGGGGAGG
AGGAGGAAGAGGTAGGAGGTAGGGGA
GGGGGCGGGGTTTTGTCACCTGTCACCTGCTCCGGCTGTGCCTAGGGCGGGCGGGCGGGGAGTGGGGGGACCG
GTATAAAGCGGTAGGCGCCTGTGCCCG
CTCCACCTCTCAAGCAGCCAGCGCCTGCCTGAATCTGTTCTGCCCCCTCCCCACCCATTTCACCACCACC
SEQ ID NO: 26 LP promoter
CTAGTACTATGCTGCACAAGCAATTTAAAACACCAACAGCAAAAAAATACACTTCTCTGAAAAAGTCTTGGTCTAG
GACCTAAACAA
TTGCCTGAAACTGGGTAGACTTACACCAATGAGAGGCAGATAAAGAGATTAAGATTGAGGGAGTAGGGCAGGGC
TTGCAATGGTGCCGGCCAGGATGTGG
CTGAGGGGGTGTGGGTGCCTGCCGTGGATGCTAGGGTAGAAGACGACTCTATTAACTGGGTGGCTGTAAGCAGT
ACCCAGGTCAATGCCTTTCATCTTCT
ACAACCTCGACGTTGCCTGGAATCCTAAATCTTTTTCTTCACTTAACAAACATCACCTCTGCTCAAATCTGCAACTGC
TTTGATATCACACTGCCTTTTT
CACCCCTCTATTATAGATGGCATTTATTTACTTACATGTTTTTTCCCCACTAGACTATACTCCTTGAGAACAGCGATT
GTGTCTTATTTATTTCTGAATCACCAATTCAGACAGGCATGCAAACACTTGT
TGAACCAATGCACAAATATATTTTGCTCTTCTTCATAGATTCCTCCGGCCTCAGATGACCAGGCACCACT
AGATACAGAACACTGTGCTTTCCTTCTCCAAGGTAAAGGAATAAATATCTGTTCCCCTTCATGAAGTGTTACTGTTG
GGCCTTTATGCCATCCTGAAGCC
ACCAGGATGTGGAACCAGATCAGGGAGGTCCACAGTTACAACCCCTTGTATCTGTAACACCAGCAGGACATTATCT
ACAGAGTCCTGCTGCAGGGCCCCG
AATGAAGACAGCATTTTGCTGCTTTGTAGCGTGAGCAGTGCTGTAACAGTGATGCATGGATGTTCCTCTGGTGTCC
TGAAAGAATGTAGGTGCTTCTTGA
AAGCTCTCTGCAACTTATTAATTGGGAGTGATTATGCGATGGAGAAAACAGAGTCCCATCACCCCCTCAGTCTTCC
CTGGGAAATCACAAGAGGGCTGAT
AGCTCTCTGTGAGGTGAACCGTTTCTAGAATCCCCACCGTCTCGTCCTGTTCTTCCGCCCACCCAGTTCCTCAAGAT
AGCCCCTGTGGGCTTCTGATGAA
GTCACCACACCACTGGCTAATGAAGTAGATAAACCAGAACAGTTTGGTTTAACATTTAAGGTCAGAAACAGGAAC
TTTCTAGAGGAGAAATCAAAAAAGC
AAAAGAAGTATAAGGGCAGCCCTCCAACCAGTCAGAATACCGTGACCACCTGAGAGGCCCGTGGCCCAGCGGAC
ACGGACGCATGTCAACTCTGGAGCAG
ATATCTTCAGCGCAGCATCTGACCTGGGAGTACAGCCACATACCCTCATTCCTAAACGGCAGATTGACTACTGC
AGCCACACACAGTCTCCGGGCAATGT
GGAGACATGTCTAATATTTAGTCAACATAACTCAGGGTGCCACAGTCTTCACAACTGTTGTGAGCACTTGAGGATG
CTGCATTTGAAGATAGGAATTTGC
CCTCAAGCATCTGGGGTTTGGGTACAGAACAGAGCTTCCCCTGCCACCACCTGCTAATTTTATAAAATGTGCATTCA
AAAAAAAATCCTGCCTGTAAGAA
GGAATTAAGCTACCCATTTAAATATAACAGCTGCCTGTGCAATCTACTGCTGCTCTTTATAGGAAACGCTTAAATAA
TTGAGATACTTAATTGGGTTAAA
GAGATCCCTAGCACATAGATGTTCTATAAATAAAAGAATGAGTAAATAATCTAGTAACCTTCCTTTTCATGTCCTTC
ACTTAAAGAGATCGTTCTGTTTT
GTTTGCACCAATAAGATCACTGTTAGAGGACTCCAGAGAGGTTTGATTTCAGGTGGGGTGGGGCTTTCCCAAGGA
AGTCCCTTTTCATTTGTTTCAGGTG
TACTGCCACCTTTTTCCCTGGCTCTTTCACTAAAAATGAAAAATTTGTTGATCTTTGCTGTAAGTAGGTAGGCATCTG
GGCTTTGCTTTTGCAACTAGAG
TCAAAGAAGTCAAGTTATCAGGCTGATCTTGCCTTGCTATCTAGAATCAGAAAGGTTTAAGTAGCCCAGGGACTAC
TCAAAGACAGCTGGAGGAGAAAGG
GAGAGAGAAAAATGCTTATAAAGAGGTGGGCAAAAGAGCGGGACCTTGTCTCAAAAAAAAAAAAAAAAAAGAG
GAAGTGGTAGGAGGTGTCTGAATTTCA
CTGTGACCTGTTCTGTCAGGTGATTTTTGGTGGGGGGGGACATGAAAAAAAAGTTAAAATGTCCTTATAAAGAC
AAAATCTTTTTCTTTCCTGGCTGAT
GATTTGTCATTCTAGTCACTTCCTGCCTTGTGACCACACACCCAGGCTTGACAAAGCTGTTCTGCAGATCAGAAAGA
AGGGGTTCCTGGTCATACACCAG TACTAC
SEQ ID NO: 27 CEACAM5 promoter
GCCCTGGAGAGCATGGGGAGA
CCCGGGACCCTGCTGGGTTTCTCTGTCACAAAGGAAAATAATCCCCCTGGTGTGACAGACCCAAGGACAGAACAC
AGCAGAGGTCAGCACTGGGGAAGAC
AGGTTGTCCTCCCAGGGGATGGGGGTCCATCCACCTTGCCGAAAAGATTTGTCTGAGGAACTGAAAATAGAAGGG
AAAAAAGAGGAGGGACAAAAGAGGC
AGAAATGAGAGGGGAGGGGACAGAGGACACCTGAATAAAGACCACACCCATGACCCACGTGATGCTGAGAAGT
ACTCCTGCCCTAGGAAGAGACTCAGGG
CAGAGGGAGGAAGGACAGCAGACCAGACAGTCACAGCAGCCTTGACAAAACGTTCCTGGAACTCAAGCTCTTCTC
CACAGAGGAGGACAGAGCAGACAGC AGAGACC
SEQ ID NO: 28 COX2 promoter
TTGAGGTACCTGGTGTAGTTTTATTTCAGGTTTTATGCTGTCATTTTCCTGTAATGCTAAGGACTTAGGACATAACT
GAATTT
TCTATTTTCCACTTCTTTTCTGGTGTGTGTGTATATATATATGTATATATACACACACACATgTACATATATATATTTT
TTAGTATCTCACCCTCACATG
CTCCTCCCTGAGCACTACCCATGATAGATGTTAAACAAAAGCAAAGATGAAATTCCAACTGTCAAAATCTCCCTTCC
ATCTAATTAATTCCTCATCCAAC
TATGTTCCAAAACGAGAATAGAAAATTAGCCCCAATAAGCCCAGGCAACTGAAAAGTAAATGCTATGTTGTACTTT
GATCCATGGTCACAACTCATAATC
TTGGAAAAGTGGACAGAAAAGACAAAAGAGTGAACTTTAAAACTCGAATTTATTTTACCAGTATCTCCTATGAAGG
GCTAGTAACCAAAATAATCCACGC
ATCAGGGAGAGAAATGCCTTAAGGCATACGTTTTGGACATTTAGCGTCCCTGCAAATTCTGGCCATCGCCGCTTCC
TTTGTCCATCAGAAGGCAGGAAAC
TTTATATTGGTGACCCGTGGAGCTCACATTAACTATTTACAGGGTAACTGCTTAGGACCAGTATTATGAGGAGAAT
TTACCTTTCCCCCCTCTCTTTCCA
AGAAACAAGGAGGGGGTGAAGGTACGGAGAACAGTATTTCTTCTGTTGAAAGCAACTTAGCTACAAAGATAAATT
ACAGCTATGTACACTGAAGGTAGCT
ATTTCATTCCACAAAATAAGAGTTTTTTAAAAAGCTATGTATGTATGTGCTGCATATAGAGCAGATATACAGCCTAT
TAAGCGTCGTCACTAAAACATAA
AACATGTCAGCCTTTCTTAACCTTACTCGCCCCAGTCTGTCCCGACGTGACTTCCTCGACCCTCTAAAGACGTACAG
ACCAGACACGGCGGCGGCGGCGG
GAGAGGGGATTCCCTGCGCCCCCGGACCTCAGGGCCGCTCAGATTCCTGGAGAGGAAGCCAAGTGTCCTTCTGCC
CTCCCCCGGTATCCCATCCAAGGCG
ATCAGTCCAGAACTGGCTCTCGGAAGCGCTCGGGCAAAGACTGCGAAGAAGAAAAGACATCTGGCGGAAACCTG
TGCGCCTGGGGCGGTGGAACTCGGGG
AGGAGAGGGAGGGATCAGACAGGAGAGTGGGGACTACCCCCTCTGCTCCCAAATTGGGGCAGCTTCCTGGGTTT
CCGATTTTCTCATTTCCGTGGGTAAA
AAACCCTGCCCCCACCGGGCTTACGCAATTTTTTTAAGGGGAGAGGAGGGAAAAATTTGTGGGGGGTACGAAAA
GGCGGAAAGAAACAGTCATTTCGTCA
CATGGGCTTGGTTTTCAGTCTTATAAAAAGGAAGGTTCTCTCGGTTAGCGACCAATTGTCATACGACTTGCAGTGA
GCGTCAGGAGCACGTCCAGGAACT
CCTCAGCAGCGCCTCCTTCAGCTCCACAGCCAGACGCCCTCAGACAGCAAAGCCTACCCCCGCGCCGCGCCCTGCC
CGCCGCTG
SEQ ID NO: 29 SURVIVIN promoter
GTTCTTTGAAAGCAGTCGAGGGGGTGCTAGGTGTGGGCAGGGACGAGCTGGCGCGGCGTCGCTGGGTGCACCG
CGACCACGGGC
AGAGCCACGCGGGGGAGGACTACAACTCCCGGCACACCCCGCGCCGCCCCGCCTCTACTCCCAGAAGGCCGCG
GGGGGTGGACCGCCTAAGAGGGCGTG
CGCTCCCGACATGCCCCGCGGCGCGCCATTAACCGCCAGATTTGAATCGCCGGACCCGTTGGCAGAGGTGGCGGC
GGCGGCA
SEQ ID NO: 30 PTP4A1_1 promoter
tccttcggctgcgggccggctcggctacgcgctctgctccgagccgctcACTGCATGGTA
SEQ ID NO: 31 SMYD5 promoter
gggaccagaagggggtgtggcctctcaggtcgaggcggggttaagggtcATAAGGCGGAG
SEQ ID NO: 32 signal peptideMEWSWVFLFFLSVTTGVHS

1. A fusion protein comprising:
(i) a first binding domain comprising:
(a) a first variable heavy domain (V_{H1}) having a sequence comprising:
(i) a vhCDR1 having at least 90% identity to SEQ ID NO: 4;
(ii) a vhCDR2 having at least 90% identity to SEQ ID NO: 5; and
(iii) a vhCDR3 having at least 90% identity to SEQ ID NO: 6; and
(b) a first variable light domain (V_{L1}) having a sequence comprising:
(i) a vlCDR1 having at least 90% identity to SEQ ID NO: 7;
(ii) a vlCDR2 having at least 90% identity to SEQ ID NO: 8; and
(iii) a vlCDR3 having at least 90% identity to SEQ ID NO: 9; and
(ii) a second binding domain comprising:
(a) a second variable heavy domain (V_{H2}) having a sequence comprising:
(i) a vhCDR1 having at least 90% identity to SEQ ID NO: 10;
(ii) a vhCDR2 having at least 90% identity to SEQ ID NO: 11; and
(iii) a vhCDR3 having at least 90% identity to SEQ ID NO: 12; and
(b) a second variable light domain (V_{L2}) comprising:
(i) a vlCDR1 comprising at least 90% identity to SEQ ID NO: 13;
(ii) a vlCDR2 comprising at least 90% identity to SEQ ID NO: 14; and
(iii) a vlCDR3 comprising at least 90% identity to SEQ ID NO: 15; and
(iii) a human Fc domain (Fc);
wherein V_{H1} and V_{L1} are connected by a first linker (L₁) and V_{H2} and V_{L2} are connected by a second linker (L₂).

2. The fusion protein as claimed in claim 1, wherein the first binding domain comprises:
(a) a first variable heavy domain (V_{H1}) having a sequence comprising:
(i) a vhCDR1 consisting of SEQ ID NO: 4;
(ii) a vhCDR2 consisting of SEQ ID NO: 5; and
(iii) a vhCDR3 consisting of SEQ ID NO: 6; and
(b) a first variable light domain (V_{L1}) having a sequence comprising:
(i) a vlCDR1 consisting of SEQ ID NO: 7;
(ii) a vlCDR2 consisting of SEQ ID NO: 8; and
(iii) a vlCDR3 consisting of SEQ ID NO: 9; and
and wherein the second binding domain comprises:
(a) a second variable heavy domain (V_{H2}) having a sequence comprising:
(i) a vhCDR1 consisting of SEQ ID NO: 10;
(ii) a vhCDR2 consisting of SEQ ID NO: 11; and
(iii) a vhCDR3 consisting of SEQ ID NO: 12; and
(b) a second variable light domain (V_{L2}) comprising:
(i) a vlCDR1 consisting of SEQ ID NO: 13;
(ii) a vlCDR2 consisting of SEQ ID NO: 14; and
(iii) a vlCDR3 consisting of SEQ ID NO; 15.

3. The fusion protein as claimed in claim 1, wherein:
the V_{H1} comprises a sequence having at least 90% identity to SEQ ID NO: 16;
the V_{L1} comprises a sequence having at least 90% identity to SEQ ID NO: 17;
the V_{H2} comprises a sequence having at least 90% identity to SEQ ID NO: 18; and
the V_{L2} comprises a sequence having at least 90% identity to SEQ ID NO: 19.

4. The fusion protein as claimed in claim 1, wherein:
(a) the V_{H1} consists of SEQ ID NO: 16;
(b) the V_{L1} consists of SEQ ID NO: 17;
(c) the V_{H2} consists of SEQ ID NO: 18; and
(d) the V_{L2} consists of SEQ ID NO: 19.

5. The fusion protein as claimed in claim 1, wherein
(a) the fusion protein further comprises a signal peptide (S), preferably wherein the signal peptide (S) is located upstream of the amino terminus of the fusion protein, even more preferably wherein the signal peptide is upstream of the amino terminus of V_{H1}; or
(b) the amino acid sequence of L₁ and L₂ are identical; optionally wherein L₁ is located downstream of the carboxy terminus of V_{H1} and upstream of the amino terminus of V_{L1} and/or wherein L₂ is located downstream of the carboxy terminus of V_{H2} and upstream of the amino terminus of V_{L2}; further optionally wherein each of L₁ and L₂ have a sequence that has at least 90% identity to SEQ ID NO: 20 or consists of SEQ ID NO: 20.

6.-8. (canceled)

9. The fusion protein as claimed in claim 1, wherein
(a) the peptide further comprises a third linker (L₃) and a fourth linker (L₄); and wherein L₃ is located downstream of the carboxy terminus of V_{L1} and upstream of the amino terminus of the Fc and L₄ is located downstream of the carboxy terminus of the Fc and upstream of the amino terminus of V_{H2}, optionally wherein each of L₃ and L₃ have a sequence that has at least 90% identity to SEQ ID NO: 21; or
(b) the human Fc domain is selected from: IgG, IgE, IgM and IgA, optionally wherein the human Fc domain is selected from: IgG1, IgG2, IgG3, and IgG4.

10.-13. (canceled)

14. The fusion protein as claimed in claim 1, wherein
(a) the human Fc domain has a sequence that is at least 90% identity to SEQ ID NO: 22 or consists of SEQ ID NO: 22;
(b) the fusion protein is arranged from amino-terminus to carboxy-terminus in an arrangement selected from: 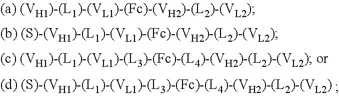
(c) the protein has a sequence that is at least 90% identical to SEQ ID NO: 23 or consists of SEQ ID NO: 23.

15.-18. (canceled)

19. A fusion protein comprising:
a first binding domain;
(b) a second binding domain; and
(c) a human Fc domain (Fc);
wherein each of the first and second binding domains selectively bind to Neu5Acα2-3Galβ1-3GalNAcα-R.

20. A nucleic acid sequence encoding the fusion protein as claimed in claim 1.

21. An expression cassette comprising a promoter operably linked to the nucleic acid according to claim 20, optionally wherein the promoter is selected from:
(i) SEQ ID NO: 25:
(ii) SEQ ID NO: 26;
(iii) SEQ ID NO: 27;
(iv) SEQ ID NO: 28;
(v) SEQ ID NO: 29;
(vi) SEQ ID NO: 3;
(vii) SEQ ID NO: 30; and
(viii) SEQ ID NO: 31.

22. (canceled)

23. An adenoviral vector comprising the expression cassette as claimed in claim 21, optionally wherein the adenoviral vector is:
(a) an oncolytic adenoviral vector; preferably a conditionally replicative adenovirus CRAd;
(b) a non-replicative adenovirus; preferably a non-replicative Ad5;
(c) a hybrid Ad5/3 adenovirus; or
(d) a hybrid Ad5/35 adenovirus.

24. (canceled)

25. An adenoviral vector comprising at least one of:
(a) the nucleic acid according to claim 20 operably linked to SEQ ID NO: 25;
(b) the nucleic acid according to claim 20 operably linked to SEQ ID NO: 26;
(c) the nucleic acid according to claim 20 operably linked to SEQ ID NO: 27;
(d) the nucleic acid according to claim 20 operably linked to SEQ ID NO: 28;
(e) the nucleic acid according to claim 20 operably linked to SEQ ID NO: 29;
(f) the nucleic acid according to claim 20 operably linked to SEQ ID NO: 3;
(g) the nucleic acid according to claim 20 operably linked to SEQ ID NO: 30;
(h) the nucleic acid according to claim 20 operably linked to SEQ ID NO: 31; and
(i) any combination of (a) to (h) above.

26. The plurality of adenoviral vectors as claimed in claim 25, wherein the plurality of adenoviral vectors comprises each of the adenovirus vectors according to claim 25 (a) to claim 25 (h).

27.-28. (canceled)

29. A method for treating a disease, wherein the method comprises: administering a therapeutically effective amount of a fusion protein as claimed in claim 1, to a patient in need thereof.

30. A method for treating cancer, wherein the method comprises: administering a therapeutically effective amount of a fusion protein as claimed in claim 1, to a patient in need thereof.

31. The method as claimed in claim 30, wherein the cancer is selected from: breast, triple-negative breast, melanoma, lung, small cell lung, B-cell leukemia, prostate, bladder, colon, glioblastoma, liver, cervical, ovarian and head and neck cancer.

32. A method for treating cancer, wherein the method comprises: administering a therapeutically effective amount of a fusion protein as claimed in claim 1, or fragments or functional variants thereof to a patient in need thereof; wherein the fusion protein, fragments, or functional variants thereof are is sequestered to the lysosome of a cancer cell, optionally wherein the sequestration to the lysosomes occurs in under 15 seconds.

33. (canceled)

34. The method of claim 32, wherein sequestration to the lysosomes leads to caspase 3-mediated apoptosis or wherein sequestration to the lysosomes leads to inhibition of sialylation.

35. (canceled)

36. The method of claim 30, wherein the fusion protein is administered at a concentration of between 1 μM and 10 μM.
